(19) United States
(12) Patent Application Publication
(10) Pub. No.: US 2018/0327703 A1
(43) Pub. Date: Nov. 15, 2018

(54) LARGE SCALE CELL MANUFACTURE SYSTEM

(71) Applicant: NUTECH VENTURES, Lincoln, NE (US)

(72) Inventor: Yuguo Lei, Lincoln, NE (US)

(21) Appl. No.: 15/777,302

(22) PCT Filed: Nov. 23, 2016
(86) PCT No.: PCT/US2016/063486

§ 371 Date: May 18, 2018

Related U.S. Application Data
(60) Provisional application No. 62/260,109, filed on Nov. 25, 2015.

(51) Int. Cl. C12M 1/12; C12N 5/00; D01D 5/24; D01F 8/18

(52) U.S. Cl. CPC C12M 25/10; C12N 5/0068; C12M 25/14; D10B 2331/14; D01F 8/18; C12N 2500/34; D10B 2509/00; D01D 5/24

(57) ABSTRACT

Methods of culturing and manufacturing of cells on a large-scale level are disclosed. Particularly, a manufacturing system and device, and methods of using the system and device for culturing and manufacturing cells in hollow fibers made from alginate polymers are provided.

### CROSS-REFERENCE TO RELATED APPLICATIONS

This application claims priority to U.S. Provisional Patent Application No. 62/260,109 filed on Nov. 25, 2015, the disclosure of which is hereby expressly incorporated by reference in its entirety.

### BACKGROUND OF THE DISCLOSURE

The present disclosure relates generally to culturing and manufacturing cells in hollow hydrogel fibers made from alginate polymers. More particularly, the present disclosure relates to a manufacturing system and device for culturing cells at various scales, particularly on a large-scale level, the cells of which can be used for various applications.

Mammalian cells have many applications. Stem cells, such as human pluripotent stem cells (hPSCs), including human embryonic stem cells (hESCs) and human induced pluripotent stem cells (iPSCs), and their progenies (i.e., cells differentiated from stem cells) can be used for treating degenerative diseases, injuries and cancers. They can also be used to make artificial tissues and organs. In addition, stem cells and their progenies can be used for modeling diseases, screening drugs and testing efficacy and toxicity of chemicals. Mammalian cells are also widely used for expressing recombinant proteins and viruses both in laboratories and industry. Many of these proteins and viruses are used in clinics. These applications require large numbers of cells of high quality. For instance, ˜10⁵ surviving dopaminergic (DA) neurons, ˜10⁹ cardiomyocytes, or ˜10⁹ β cells are required to treat a patient with Parkinson's disease (PD), myocardial infarction (MI), or Type 1 diabetes, respectively. Additionally, far more cells are needed initially because both in vitro cell culture yields and subsequent in vivo survival of transplanted cells are typically very low. As examples of the latter, only ˜6% of transplanted dopaminergic neurons or ˜1% of injected cardiomyocytes reportedly survived in rodent models several months after transplantation. Furthermore, there are large patient populations with degenerative diseases or organ failure, including over 1 million people with PD, 1-2.5 million with Type 1 diabetes, and ˜8 million with MI in the US alone. Large numbers of cells are also necessary for applications such as tissue engineering, where, for example, ˜10¹⁰ hepatocytes or cardiomyocytes would be required for an artificial human liver or heart, respectively. Additionally, ˜10¹⁰ cells may be needed to screen a million-compound library once, and advances in combinatorial chemistry, noncoding RNAs, and investigations of complex signaling and transcriptional networks have given rise to large libraries that can be screened against many targets. Large numbers of mammalian cells, such as Chinese Hamster Ovary cells (CHO cells) and Human Embryonic Kidney 293 cells (HEK293), are also needed for producing therapeutic biologics, such as monoclonal antibodies (mAbs), enzymes and viral particles.

Currently, there are few methods that can cost-effectively manufacture stem cells, and their progenies, and primary cells, especially in large scale. The most widely used 2D cell culture systems, in which cells are cultured on a 2D surface, are limited by their low yield, heterogeneity, scalability and reproducibility. For instance, only about 50,000 cardiomyocytes can be cultured per cm² of surface area.

Due to the above drawbacks, three dimensional (3D) suspension cell culture systems, such as spinner flasks and stirred-take bioreactors are being widely studied to scale up the production. However, cellular spheroids in suspension cultures frequently aggregate to form large cellular agglomerates. It is well known that the transport of nutrients, oxygen and growth factors to, and the metabolic waste from cells located at the center of agglomerates (FIG. 10A) with diameters larger than 500 μm become insufficient, leading to slow cell proliferation, apoptosis, and uncontrolled differentiation. While stirring or shaking the culture reduces cellular agglomeration, they also generate hydrodynamic stress that negatively affects cell viability, proliferation and phenotype. High cell density in the culture also promotes cellular agglomeration. Considering all these factors, in current suspension culture studies, cells are generally seeded at low density (e.g., ˜3×10⁵ cells/mL) and stirred at 70 to 120 rotations-per-minute (rpm). Under even these optimized conditions, slow cell growth, significant cell death, phenotype change, genomic mutations, and low volumetric yield are common. For instance, it has been shown that hPSCs typically expanded 4-fold per 4 days to yield around 2.0×10⁶ cells/mL. These cells merely occupy less than 0.4% of the bioreactor volume. The low yield leads to both economic and technical challenges for manufacturing large-scale cells.

Based on the foregoing, there is a need in the art for a robust cell culture system that can cost-effectively manufacture different types of cells at various scales, particularly at large scale. This system would be useful in both research laboratories and industry.

### BRIEF DESCRIPTION OF THE DISCLOSURE

The present disclosure is generally directed to culturing and manufacturing mammalian cells in hollow hydrogel fibers made from alginate polymers. More particularly, the present disclosure is directed to a culturing system and device capable of manufacturing cells at various scales, especially at large-scale levels, and to methods of using the system and device for culturing and manufacturing cells in hollow hydrogel fibers made from alginate polymers.

It has been found that use of the hollow hydrogel fibers as a cell culture system promotes initial cellular clustering, ensures efficient mass transport to cells and eliminates hydrodynamic stress for cells, allows culturing cells with high viability, high cell growth rate and high volumetric yield (e.g. producing up to 5.0×10⁸ cells per milliliter of volume). These advantages dramatically reduce the bioreactor volume, production time and cost. Thus, this new culture system has potential to transform the cellular manufacturing.

In one aspect, the present disclosure is directed to a method of manufacturing cells at various scales, the method comprising: suspending a cell solution including cells in the hollow space of alginate hydrogel fibers; and suspending the hollow fibers including the cells in cell culture medium; and culturing the cells.

In another aspect, the present disclosure is directed to a method of manufacturing cells at various scales, the method comprising: extruding a cell solution and an alginate solution into a cell compatible solution, the cell compatible solution crosslinking the alginate polymers within the alginate polymer solution to form hollow alginate hydrogel fibers; suspending the hollow fibers including the cells in cell culture medium or cell compatible buffer; and culturing the cells.

In another aspect, the present disclosure is directed to a system for culturing cells. The system comprising: a housing comprising a first inlet, a second inlet, a core channel, a shell channel, and an outlet, the first inlet operable for introducing a cell solution into the core channel, the second inlet operable for introducing an alginate solution into the shell channel, wherein the shell channel is in fluid contact with the core channel to allow contact between the cell solution and alginate solution, and a cell culture vessel in fluid contact with the housing at the outlet, wherein the cell culture vessel comprises a cell compatible buffer.

In accordance with the present disclosure, methods have been discovered that surprisingly allow for culturing various types of cells on a large-scale level. As used herein, “large” or “large-scale” refers to a product of from about 10⁷ to about 10³⁰ cells, including from about 10⁷ to about 10¹⁵ cells, and including from about 10⁷ to about 10¹² cells. The methods and manufacturing system of the present disclosure will have significant impact on regenerative medicine as they allow for sufficient, high quality and affordable cells. Further, the system and methods provide an advantageous impact on the biopharmaceutical industry by providing more affordable methods for manufacturing recombinant proteins and viruses.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a schematic depicting a device of the present disclosure for processing hollow alginate hydrogel fibers.

FIG. 2 depicts an exemplary device of the present disclosure for processing hollow alginate hydrogel fibers.

FIGS. 3A-3E is a schematic depicting the method steps of the present disclosure for culturing cells within the hollow alginate hydrogel fibers. FIG. 3A depicts cells cultured in a medium-filled space of hollow alginate hydrogel fibers. The fibers including the cells are suspended in cell culture medium in cell culture vessels or bioreactors. Cells are expanded (FIG. 3B) and harvested (FIG. 3C) or differentiated (FIG. 3D) in the hollow fibers. Cells in the hollow fibers can also be used to produce recombinant proteins and viruses (FIG. 3E).

FIG. 4 depicts hollow alginate hydrogel fibers including cells suspended in a cell culture medium as disclosed in the present disclosure.

FIGS. 5A-5C depict culturing stem cells in hollow hydrogel fibers. H9 Human embryonic stem cells (FIG. 5A), induced human pluripotent stem cells: MSC-iPSCs (iPSCs made from human mesenchymal stem cells) (FIG. 5B) and Fib-iPSCs (iPSCs made from human fibroblasts) (FIG. 5C) are shown. Cells were cultured in the hollow fibers for 8 days, during which cells grew into large aggregates from single cells.

FIGS. 6A-6F depict human iPSCs differentiated into cortical neurons (FIGS. 6A-6C) and dopaminergic neurons (FIGS. 6D-6F). FIGS. 6A and 6B depict phase images of cortical neurons within the hollow alginate hydrogel fibers at day 30. FIGS. 6D and 6E depict phase images of dopaminergic neurons within the hollow fibers at day 30. FIGS. 6C and 6F depict immunostaining at day 30 of human iPSCs differentiated into corresponding neurons.

FIGS. 7A-7C depicts human glioblastoma stem cells cultured in hollow alginate hydrogel fibers. FIG. 7A depicts cell line L0 cultured in the hollow fibers over a period of 7 days. FIG. 7B depicts cell line L1 cultured in the hollow fibers over a period of 7 days. FIG. 7C depicts cell line L2 cultured in the hollow fibers over a period of 7 days. Cells grew into large aggregates from single cells.

FIG. 8 depicts mouse L cells cultured in hollow alginate hydrogel fibers for producing recombinant Wnt 3A proteins. L cells stably expressing Wnt3A proteins were cultured in the hollow fibers for 6 days. Cells grew into high density aggregates by day 6.

FIGS. 9A-9J depicts the hollow alginate hydrogel fiber cell culture system (“cell culture system)” as analyzed in Example 5. FIGS. 9A & 9B show a home-made micro-extruder for processing one hollow fiber. A hyaluronic acid (HA) solution containing single cells and an alginate solution was pumped into the central and side channel of the micro-extruder, respectively, to form a coaxial core-shell flow that is extruded into a 100 mM CaCl₂ buffer, which instantly crosslinks the alginates to form a hydrogel shell to make one hollow fiber. Subsequently, CaCl₂ buffer is replaced by cell culture medium and cells are suspended and grown in the core microspace of the hollow fiber. FIG. 9C depicts freshly prepared hollow fibers in the CaCl₂ buffer. FIGS. 9D-9F depict a micro-extruder with 9 nozzles for simultaneously processing 9 hollow fibers. FIG. 9G depicts that HAs are required to process defect-free hollow fibers. Without HAs (−HA), fibers with asymmetric shells or beads are formed. FIG. 9H is an illustration of a hollow alginate hydrogel fiber showing cell growth in the cell culture system. Within hours, single cells associate to form small cell clusters (i.e. the initial clustering). Subsequently, cells proliferate and the small cell clusters expand as spheroids that eventually merge to form a cylindrical cell mass. The diameter of the cell mass is controlled to be less than 500 μm to ensure efficient mass transport in the cell mass. Two vials of H9 hESCs, stained with DIO and DID dyes appearing green and red fluorescence, respectively, were mixed at 1:1 and cultured in the cell culture system. Single cells (day 0), small cell clusters (day 1), a cylindrical cell mass (day 9) were clearly seen. FIG. 9J depicts ROCK inhibitors (RIs) required for the initial cell survival. Live/dead staining showed a majority of the cells went apoptosis after 24 hours without RIs (−RI). Cells survived and grew well with RIs (+RI). Scale bar: (FIGS. 9G, 9I, and 9J) 200 μm.

FIG. 10A depicts that in the current 3D suspension cultures (e.g. spinner flasks or stirred-tank bioreactors), single hPSCs associated to form small cell clusters within 24 hours (i.e. the initial clustering phase) that subsequently expanded as spheroids (i.e. the cell expansion phase). Cells and spheroids frequently fused to each other to form large agglomerates. FIG. 10B confirms cellular agglomeration in experiment: two vials of H9 hESCs, stained with DIO and DID dyes respectively, were mixed at 1:1 and cultured in suspension. The lipophilic DIO and DID dyes stained cells to appear green and red, respectively, under fluorescent microscopy. Single cells (day 0), small clusters with both green and red cells (day 1), spheroids and agglomerates with both green and red cells (day 4) were clearly seen. Scale bar: 100 μm.

FIGS. 11A-11F depict the influence of alginate hydrogel formulation on hPSC culture in the cell culture system. H9 hESCs were cultured for 9 days in hollow alginate hydrogel fibers (inner diameter: ˜400 μm; shell thickness: ˜40 μm) processed from 2% alginates from Sigma (#A2033-100G) or Wako Chemicals with varied viscosity or molecular weight (500˜600 cp; 300˜400 cp and 80˜120 cp). FIG. 11A depicts phase images showing single H9s on day 0 and H9 spheroids on day 4 in the hollow fibers. FIG. 11B depicts that live/dead staining revealed almost no dead cells in the hollow fibers. FIG. 11C depicts Oct4 staining on day 10 cells. H9s were released from hollow fibers on day 9 and plated on Matrigel-coated plate overnight before fixing and staining. Arrows point to the differentiated Oct4− cells. FIGS. 11D & 11E depict expansion fold and volumetric yield on day 5, 7 and 9. FIG. 11F depicts the % of Oct4+ cells after the 9-day culture. Error bars represent the standard deviation (n=3). *** indicates statistical significance at a level of p<0.001. Scale bar: (FIGS. 11A & 11B) 400 μm; (FIG. 11C) 50 μm.

FIGS. 12A-12E depict the influence of alginate hydrogel formulation on hPSC culture in the cell culture system. H9s were cultured for 9 days in hollow alginate hydrogel fibers (inner diameter: ˜400 μm; shell thickness: ˜40 μm) processed from 1%, 1.5% or 2% alginates from Wako Chemicals (80˜120 cp). FIG. 12A depicts phase images of the day 0, 1 and 8 cells in hollow fibers. FIGS. 12B & 12C depict expansion fold and volumetric yield on day 5, 7 and 9. FIG. 12D depicts Oct4 staining on day 10 cells. H9s were released from hollow fibers on day 9 and plated on Matrigel-coated plate overnight before fixing and staining. FIG. 12E depicts the % of Oct4+ cells after the 9-day culture. Error bars represent the standard deviation (n=3). Scale bar: (FIG. 12A) 400 μm; (FIG. 12D) 50 μm.

FIGS. 13A-13G depict the influence of hydrogel shell thickness on hPSC culture in the cell culture system. H9s were cultured for 9 days in hollow alginate hydrogel fibers with shell thickness of 30, 40, 70, or 90 μm processed from 1.5% alginates from Wako Chemicals (80˜120 cp). FIG. 13A gives the equation used to predict the shell thickness based on the volumetric flow rates of the cell solution and alginate solution and the fiber outer diameter. FIG. 13B depicts that the experimental shell thickness fit well with the predicted data. FIG. 13C depicts phase images of the cells in hollow fibers with varied shell thickness on day 0. FIGS. 13D & 13E depict expansion fold and volumetric yield on day 5, 7 and 9. FIG. 13F depicts Oct4 staining on day 10 cells. H9s were released from hollow fibers on day 9 and plated on Matrigel-coated plate overnight before fixing and staining. FIG. 13G depicts the % of Oct4+ cells after the 9-day culture. Error bars represent the standard deviation (n=3). Scale bar: (FIG. 13C) 200 μm; (FIG. 13D) 50 μm.

FIGS. 14A-14E depict the influence of hollow fiber inner diameter on hPSC culture in the cell culture system. H9s were cultured for 9 days in hollow alginate hydrogel fibers with inner diameter of 400, 250 or 120 μm processed from 1.5% alginates from Wako Chemicals (80˜120 cp). FIG. 14A depict phase images of the day 0, 1, 5 and 8 cells in hollow fibers. FIGS. 14B & 14C depict expansion fold and volumetric yield on day 5, 7 and 9. FIG. 14D depicts Oct4 staining on day 10 cells. H9s were released from hollow fibers on day 9 and plated on Matrigel-coated plate overnight before fixing and staining. FIG. 14E depicts the % of Oct4+ cells after the 9-day culture. Error bars represent the standard deviation (n=3). Scale bar: (FIG. 14A) 400 pm; (FIG. 14D) 50 pm.

FIGS. 15A-15F depict the influence of the liquid core niche on hPSC culture in the cell culture system. H9s were cultured for 9 days in hollow alginate hydrogel fibers processed from 1.5% alginates from Wako Chemicals (80˜120 cp) with varied core liquid formulations including 3% methylcellulose (MC), 1% hyaluronic acid (HA), 2% HA, 2% HA+1 μg/mL fibronectin+0.5 μg/mL laminin or 2% HA+StemBeads. FIG. 15A depicts phase images showing day 0 and day 3 cells. FIG. 15B depicts that live/dead staining revealed almost no dead cells in the hollow fibers. FIG. 15C depicts Oct4 staining on day 10 cells. H9s were released from hollow fibers on day 9 and plated on Matrigel-coated plate overnight before fixing and staining FIGS. 15D & 15E depict expansion fold and volumetric yield on day 5, 7 and 9. FIG. 15F depicts the % of Oct4+ cells after the 9-day culture. Error bars represent the standard deviation (n=3). Scale bar: (FIGS. 15A & 15B) 400 μm; (FIG. 15C) 50 μm.

FIGS. 16A-16E depict the influence of cell seeding density on hPSC culture in the cell culture system. H9s were cultured for 9 days in hollow alginate hydrogel fibers processed from 1.5% alginates from Wako Chemicals (80˜120 cp). FIG. 16A depicts phase images of the cells in hollow fibers. After 24 hours, the cell clusters were bigger at higher seeding density, but the number of clusters were similar. FIG. 16B depicts that the expansion fold on day 5, 7 and 9 showed hPSCs grew faster at lower seeding density, while the final volumetric yields on day 9 were very close (FIG. 16C). FIG. 16D depicts Oct4 staining on day 10 cells. H9s were released from hollow fibers on day 9 and plated on Matrigel-coated plate overnight before fixing and staining. FIG. 16E depicts the % of Oct4+ cells after the 9-day culture. Error bars represent the standard deviation (n=3). *** indicates statistical significance at a level of p<0.001. Scale bar: (FIG. 16A) 400 μm; (FIG. 16D) 50 μm.

FIGS. 17A-17F depict culturing hPSCs in the cell culture system with ultralow seeding densities. H9s were seeded at 1.0×, 3.0× or 5.0×10⁵ cells/mL in hollow alginate hydrogel fibers processed from 1.5% alginates from Wako Chemicals (80˜120 cp). FIG. 17A depicts phase images showing a few H9s grew into cylindrical cell mass in the hollow fibers. FIG. 17B depicts that live/dead staining revealed almost no dead cells. FIG. 17C are images showing a single fiber with H9s on varied days along the culture. FIG. 17D depicts that the final volumetric yields were close at all seeding densities. FIG. 17E depicts Oct4 staining on day 10 cells. H9s were released from hollow fibers and plated on Matrigel-coated plate overnight before fixing and staining. FIG. 17F depicts the % of Oct4+ cells after 10-day culture. Error bars represent the standard deviation (n=3). Scale bar: (FIGS. 17A & 17B) 400 μm; (FIG. 17E) 50 μm.

FIGS. 18A-18D depict the passage 1 culturing of hPSCs in the cell culture system. H9s, MSC-iPSCs and Fib-iPSCs were cultured in hollow alginate hydrogel fibers processed from 1.5% alginates from Wako Chemicals (80˜120 cp). FIG. 18A depicts phase images and live/dead staining of hPSCs in the cell culture system. FIGS. 18B & 18C depict expansion fold and volumetric yield on day 5, 7 and 9. FIG. 18D depict that the day 9 cell mass was fixed and stained for the pluripotency markers: Nanog, Oct4, SSEA-4 and alkaline phosphatase (ALP). Images of varied slices of a cylindrical cell mass were shown. Similar results were obtained for MSC-iPSCs and Fib-iPSCs. Error bars represent the standard deviation (n=3). Scale bar: 400 μm.

FIGS. 19A-19G depict long-term culturing of hPSCs in the cell culture system. H9s, Fib-iPSCs and MSC-iPSCs were cultured in hollow alginate hydrogel fibers processed from 1.5% alginates from Wako Chemicals (80˜120 cp) for 10 passages. FIG. 19A depicts phase images of day 0, 3, and 5 cells in hollow fibers at passage 10. FIG. 19B depicts that live/dead staining revealed almost no dead cells in the hollow fibers at passage 10. FIGS. 19C & 19D depict expansion fold and volumetric yield on day 5, 7 and 9 of hPSCs at passage 10. FIG. 19E depicts the expression of the pluripotency markers: Nanog, Oct4, SSEA-4 and alkaline phosphatase (ALP) in the day-9 cell mass at passage 10. FIG. 19F shows ˜95% of the passage 10 cells expressed Oct4 and Nanog. FIG. 19G depicts that when seeded at 1.0×10⁷ cells/mL, hPSCs consistently expanded ˜15-fold per passage per 5 days during the long-term culture. Error bars represent the standard deviation (n=3). Scale bar: (FIGS. 19A & 19B) 400 μm; (FIG. 19E) 200 μm.

FIGS. 20A-20F show that hPSCs retained pluripotency after long-term culturing in the cell culture system. H9s were cultured in hollow alginate hydrogel fibers processed from 1.5% alginates from Wako Chemicals (80˜120 cp) for 10 passages. Cells were differentiated into the Nestin+ ectodermal, α-SMA+ mesodermal and FOXA2+ endodermal cells in the embryoid assay (EB) assay (FIG. 20A), formed teratomas containing the three germ layer tissues (FIG. 20B) and had normal karyotypes (FIG. 20C). By further culturing in a mesodermal (FIG. 20D) or endodermal (FIG. 20E) or cardiomyocyte (FIG. 20F) differentiation medium, hPSCs in the hollow alginate hydrogel fibers could be differentiated into the corresponding Brachyury+ mesodermal cells or FOXA2+ endodermal cells or cTNT+ cardiomyocytes at high efficiency. Scale bar: (FIGS. 20A & 20B) 100 μm; (FIGS. 20D-20F) 200 μm.

FIGS. 21A-21F depict hPSCs retained pluripotency after long-term culturing in the cell culture system. MSC-iPSCs and Fib-iPSCs were cultured in hollow alginate hydrogel fibers processed from 1.5% alginates from Wako Chemicals (80˜120 cp) for 10 passages. Both cells were differentiated into the Nestin+ ectodermal, α-SMA+ mesodermal and FOXA2+ endodermal cells in the EB assay (FIGS. 21A & 21B), formed teratomas containing the three germ layer tissues (FIGS. 21C & 21D) and had normal karyotypes (FIGS. 21E & 21F). Scale bar: 100 μm.

FIG. 22 depicts hPSCs retained pluripotency after long-term culturing in the cell culture system. H9s, MSC-iPSCs and Fib-iPSCs were cultured in hollow fibers processed from 1.5% alginates from Wako Chemicals (80˜120 cp) for 10 passages. These cells were further cultured on Matrigel-coated plates. Images of the hPSC colonies expressing the pluripotency marker Oct4 after one passage on Matrigel-coated plates are shown. Scale bar: 100 μm.

FIGS. 23A-23F depict a prototype bioreactor with the hollow alginate hydrogel fibers. FIG. 23A depicts hollow fibers with cells suspended in a cylindrical container. Medium was stored in a plastic bellow that could be pressed to flow the medium into or released to withdraw the medium from the container, respectively. FIG. 23B shows images of the mechanic stage for pressing and releasing the bellow; the controller that can be programmed for the pressing and releasing speed as well as the duration of the interval between the pressing and releasing; and the container and bellow. FIG. 23C is an image of the cylindrical, white cell mass in the bioreactor on day 10. FIG. 23D shows that 1.0×10⁹ cells were produced with 2.0 mL hollow fibers. FIGS. 23E & 23F show that these cells expressed the pluripotency markers: Nanog, Oct4, SSEA4 and ALP. Error bars represent the standard deviation (n=3). Scale bar: (FIG. 23C) 1 cm; (FIG. 23E) 200 μm.

FIGS. 24A-24E depict culturing L-Wnt-3A-cells engineered to express Wnt3a proteins in the cell culture system. Cells were cultured in the cell culture system with seeding density at 1.0× or 2.0×10⁷ cells/mL. FIG. 24A depicts phase images of cells in hollow fibers. FIG. 24B depicts that live/dead staining revealed almost no dead cells. FIGS. 24C & 24D depict expansion fold and volumetric yield on day 2 to 6. FIG. 24E shows that Wnt3a proteins were consistently expressed during a 16-day culture. Error bars represent the standard deviation (n=3). Scale bar: (FIGS. 24A & 24B) 400 μm.

FIGS. 25A-25E depicts a prototype bioreactor for the cell culture system. Hollow fibers with cells were contained a closed cell culture chamber. Medium was stored in a flask and continuously perfused into the chamber. FIG. 25C is an image of the cylindrical, white cell mass in (harvested from the bioreactor on day 10) a 10 cm dish. FIGS. 25D & 25E depicts an extruder with 100 nozzles for simultaneously processing 100 hollow fibers.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In accordance with the present disclosure, methods have been discovered that surprisingly allow for the culturing and manufacturing of cells on a large-scale level. Particularly, the present disclosure provides a manufacturing system and device, and methods of using the system and device for culturing and manufacturing cells in hollow fibers made from alginate polymers.

### Methods of Manufacturing/Culturing Cells

The methods of the present disclosure may be used to culture and manufacture cells at various scales. The methods provide at least the following advantages over conventional cell culture methods: (1) allow for large-scale cell manufacture; (2) allow for high density cell culture, thereby reducing the space, labor, and materials of cell culture; (3) allow for culturing various types of cells; and (4) allow for manufacturing cells in a much cheaper, more efficient manner Non-limiting examples of such cells that can be cultured and manufactured using the methods and systems described herein include mammalian cells, insert cells (e.g., drosophila S2 cells), plant cells, yeast cells, and bacterial cells. While described more fully using mammalian cells, especially human pluripotent stem cells, it should be recognized that the methods and systems described herein can be used with any of the above-listed types of cells without departing from the scope of the present disclosure.

As used herein, “mammalian cells” refer to cells derived from both humans and animals. Particularly suitable mammalian cells for use in the methods and systems of the present disclosure include, mammalian embryonic stem cells, mammalian induced pluripotent stem cells, mammalian naive pluripotent stem cells, cells differentiated from mammalian embryonic stem cells, mammalian induced pluripotent stem cells and mammalian naive pluripotent stem cells, mammalian cells reprogrammed from other cell types (e.g. human neurons reprogrammed from human fibroblasts), mammalian primary cells (e.g., human umbilical vein endothelial cells, cancer cells, T cells), mammalian tissue stem cells (e.g., mesenchymal stem cells, fetal neural stem cells), mammalian cell lines (e.g., human embryonic kidney (HEK293) cells, Chinese hamster ovary (CHO) cells).

In general, the method of the present disclosure includes: suspending cells in a liquid medium-filled space within hollow hydrogel fibers; suspending the hollow fibers in a cell culture medium to allow expansion and/or differentiation of the cells; and harvesting the cells.

The cells are suspended in a cell culture medium or cell compatible buffer to form a cell solution. The cell culture medium is cell type dependent. Suitably, cells are suspended in medium at concentrations varying from 1 to a few billion cells per cubic milliliter.

The hollow fibers are prepared from alginate polymer material. Suitable alginate polymer material for use in preparing the fibers include any commercially available or home-purified alginate polymer, such as alginate acid or sodium alginate from Sigma (+W201502), and modified alginate polymers, such as methacrylate modified alginate, and combinations thereof. As used herein, “combinations thereof” refer to mixtures of the polymers as well as polymer blends. Further, in some embodiments, other polymers such as hyaluronic acids can be blended or incorporated into the alginate polymers to dope the alginate hydrogel. To form the fibers, alginate polymers are first dissolved in water or cell compatible buffer to form alginate solutions including from about 0.01% (w/v) to about 20% (w/v) alginate. In particularly suitable aspects, the fibers are then prepared and filled with cells using an extruder. Extrusion conditions will be those known in the art suitable for the particular cell survival and growth.

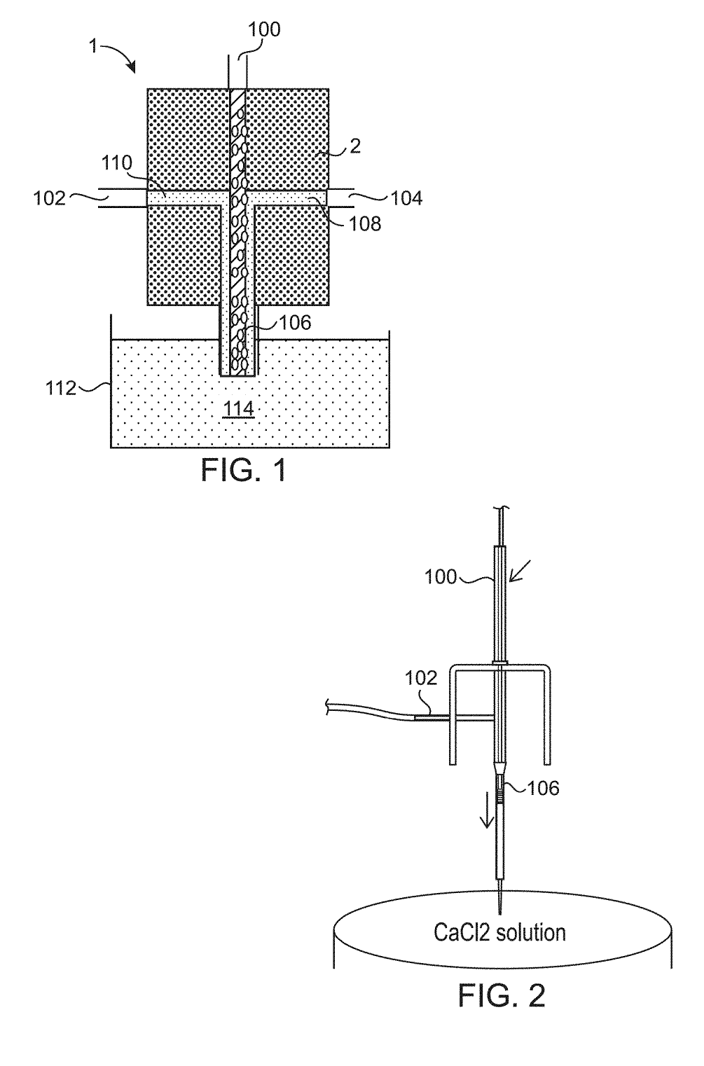

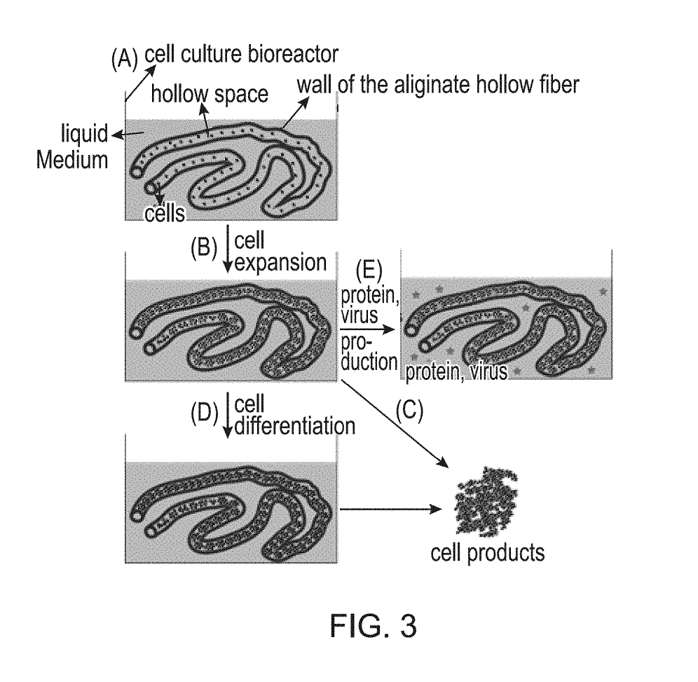

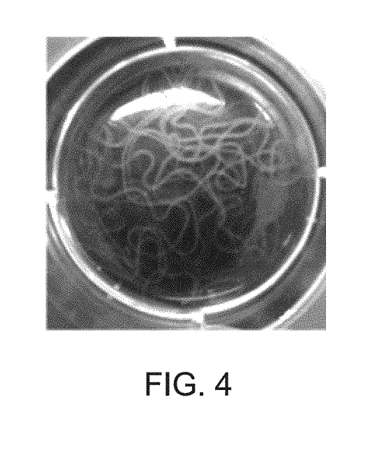

By way of example, as shown in FIGS. 1 and 2, a cell solution including cells is supplied via a first inlet 100 and the alginate solutions are supplied via at least a second inlet (shown in FIG. 1 as inlets 102, 104). Both the first stream including the cell solution and the second stream including the alginate solution are extruded into a cell compatible solution containing calcium ions or other ions or chemicals, such as barium ions, that can crosslink the alginate polymers in the alginate solution. The cell compatible solution allows the alginate polymers to instantly crosslink, thereby gelling the alginate solution and forming the hollow fibers. Typically, the fibers are sufficiently crosslinked in a time period of from about one minute to about 30 minutes.

Typically, as formed, the hollow fibers will be sized for the particular cells and amount of cell expansion desired. The fibers can have a length typically ranging from millimeters to meters. Additionally, the outer and inner diameters of the hollow hydrogel fibers can vary from micrometers to millimeters.

Once sufficiently crosslinked to form hollow fibers, the cell compatible solution is removed and cell culture medium is added to culture the cells now within the crosslinked hollow alginate hydrogel fibers. In some aspects, the fibers, including cells, are suspended in cell culture medium in cell culture vessels or bioreactors. The cell culture medium can be any medium known in the cell culture art that is suitable for supporting cell survival, growth and differentiation. Typically, the cell culture medium will include, but is not limited to, a carbon source, a nitrogen source, and growth factors. The specific cell culture medium for use in culturing the cells within the crosslinked hollow alginate hydrogel fibers will depend on the cell type to be cultured.

Cell culture conditions will vary depending on the type of cell, the amount of cell expansion, and the number of cells desired. Once sufficient cell expansion and desired numbers of cells are reached, the cells can be passaged and seeded into new crosslinked hollow alginate hydrogel fibers for a subsequent round of growth and expansion. Alternatively, the expanded cells can be differentiated into the final desired cell type within the hollow space.

Cells are finally released from the hollow space of the fiber by dissolving the fiber chemically or physically. In one aspect, the fiber is dissolved using a chemical dissolvent such as ethylenediaminetetraacetic acid (EDTA), ethylene glycol tetraacetic acid (EGTA), or an alginate lyase solution (available from Sigma-Aldrich). In another aspect, the fiber is dissolved using a mechanical force. The duration of the cells within the hollow fiber can typically vary from days to months.

The cells are useful in both research laboratories and industry. Small scale and large scale of cells can be manufactured with the system for laboratorial and industrial applications, respectively. Cells can be efficiently and effectively prepared in size and number for use in degenerative disease and injury treatment, drug screening, for expressing proteins and the like. Additionally, the cells can be used to manufacture proteins and vaccines. In yet other aspects, the cells can be used for tissue engineering.

### System/Device for Processing Alginate Hollow Fibers

In another aspect, the present disclosure is directed to a device for processing hollow fibers from alginate polymers with cells suspended in the hollow space. Generally, referring to FIG. 1, the device 1 includes a housing 2 including a core channel 106 running down the center of the housing 2. The core channel connects to the first inlet 100 for introducing cells into the housing 2. The housing 2 of the device 1 further includes shell channels 108, 110 for flowing alginate solution introduced through the second inlets 104, 102 into the housing 2. Although shown with two shell channels, it should be understood that the housing may include less or more shell channels, such as a single shell channel, or three, four, five or more shell channels, without departing from the scope of the present disclosure. In some particularly suitable embodiments, pumps (not shown) are included at the inlets 100, 102, 104 for pushing streams of cells and alginate solution into the housing 2 of the device 1.

The outlet of channel 106 of the device 1 is in contact with a cell culture vessel or bioreactor 112 including cell compatible buffer to form a system including the housing 2 and the cell culture vessel or bioreactor 112. The vessel 112 includes a buffer 114 as described above including calcium ions or other ions or chemicals that can crosslink the alginate polymers within the alginate solution to gel the solution to form the fibers.

The disclosure will be more fully understood upon consideration of the following non-limiting Examples.

### EXAMPLES

Unless otherwise indicated, the hollow fibers were prepared as described above.

### Example 1

In this Example, expansion and growth of human pluripotent stem cells, including human embryonic stem cells (hESCs) and human induced pluripotent stem cells (human iPSCs) in hollow fibers were analyzed over 8 days.

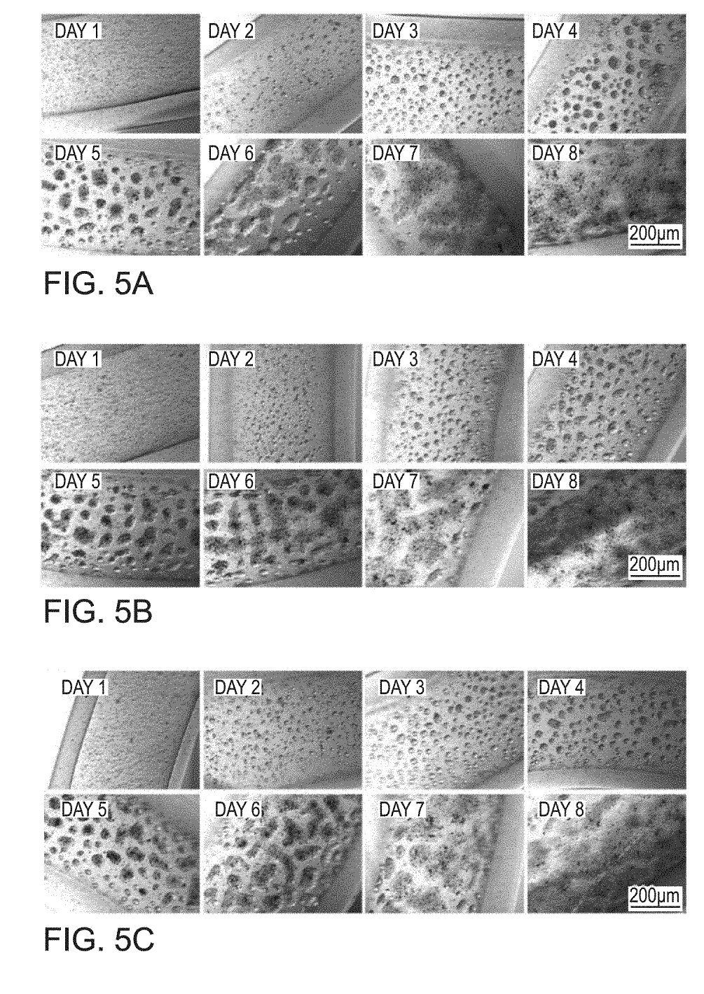

Single human embryonic stem cells (H9, WiCell) (FIG. 5A) or induced human pluripotent stem cells reprogrammed from human mesenchymal stem cells (MSC-iPSCs) (FIG. 5B) or from human skin fibroblasts (Fib-iPSCs) (FIG. 5C) were suspended in Essential 8 Medium (Life Technology) containing 0.5% (w/v) hyaluronic acid (Lifecore Biomedical) at a density of 1×10⁶ cells/ml. Sodium alginate was dissolved in 0.9% (w/v) saline to reach a concentration of 1.2% (w/v) alginate and autoclaved. With an extruder (see e.g., FIGS. 1 and 2), 10 ml of cell solution and 10 ml of alginate solution were extruded into the 100 ml of sterile buffer containing 100 mM CaCl₂ at room temperature to form alginate hollow fibers with cells suspended in the hollow space. The fibers were crosslinked in the CaCl₂ solution for 5 minutes at room temperature. The CaCl₂ solution was removed and replaced with Essential 8 Medium. Cells were cultured in the hollow fibers suspended in the medium in a regular cell culture incubator at 37° C., with 5% CO₂, 95% air at 1 atm for 8 days. Single cells grew into cell aggregates. To harvest cells, the Essential 8 Medium was removed and replaced with PBS containing 100 mM EDTA (Sigma) or 40 mg/ml alginate lyase (Sigma) at 37° C. for 10 minutes. The alginate hydrogel fibers were dissolved and cells were harvested. These cell aggregates can be dissociated into single cells by treating them with Accutase (Life Technology) at 37° C. for 10 minutes. Cells can be processed into the alginate hollow fibers for a second round of expansion. As shown in FIGS. 5A-5C, the cells grew into large aggregates from single cells effectively using the hollow fibers.

### Example 2

In this Example, hollow alginate fibers including human iPSCs as made in Example 1 were differentiated into cortical neurons and dopaminergic neurons within the fibers.

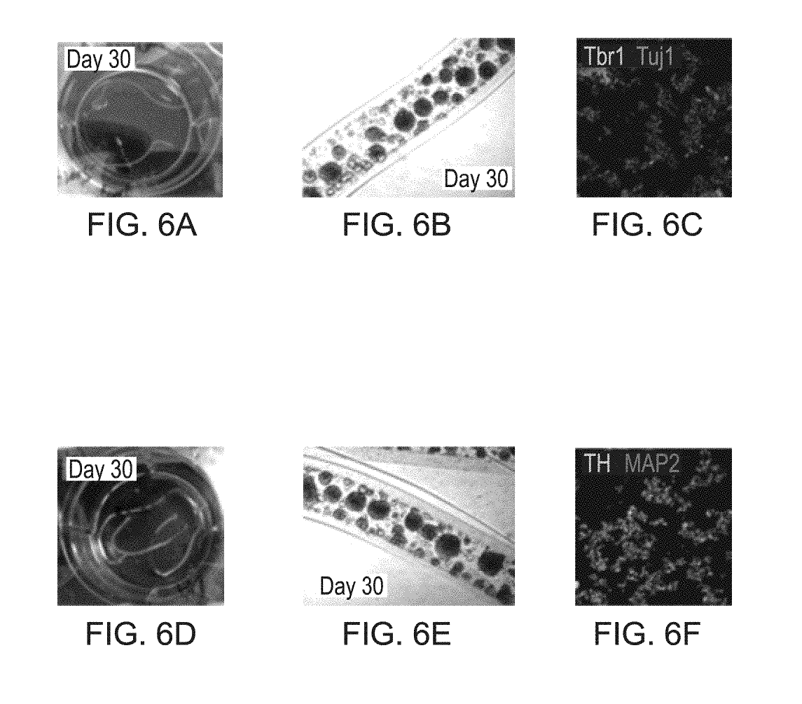

Human MSC-iPSCs were allowed to expand in the hollow fibers for 5 days. The Essential 8 Medium was then replaced with home-made and chemically defined neuronal differentiation mediums and then differentiated into cortical and dopaminergic neurons within the alginate hollow fibers for 30 days. Results are shown in FIGS. 6A-6F. As shown in FIGS. 6C and 6F, immunostaining on day 30 indicated that the majority of human iPSCs were differentiated into corresponding neurons.

### Example 3

In this Example, human glioblastoma stem cells were cultured in hollow fibers.

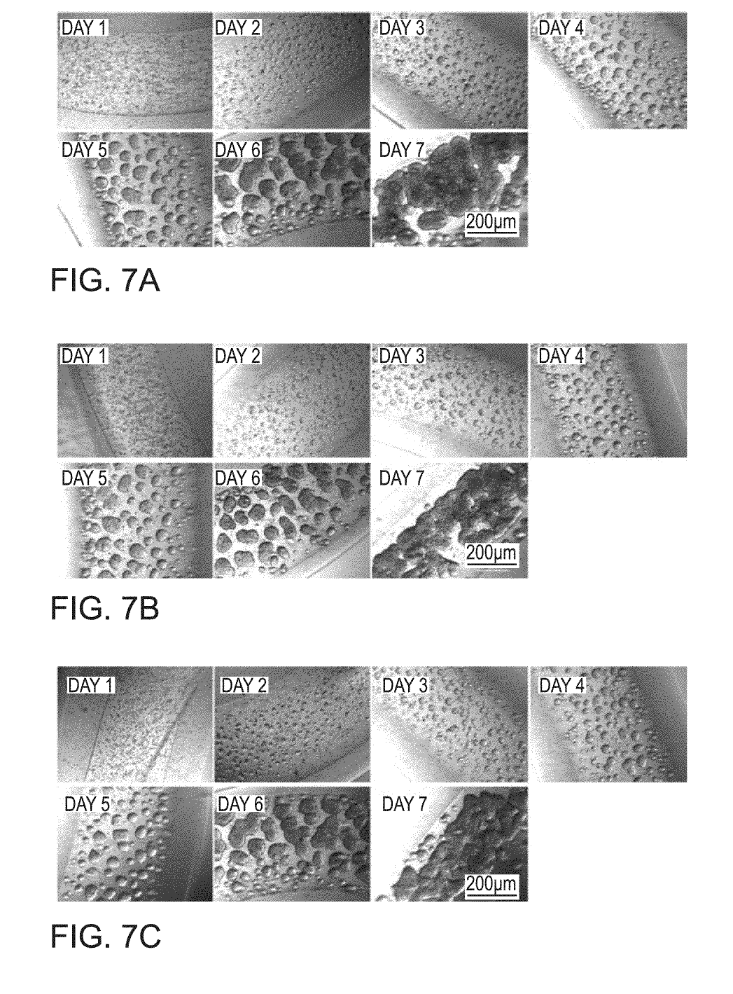

Three cancer stem cell lines, L0, L1 and L2, isolated from human glioblastoma were cultured in the hollow fibers. Single cells were suspended in NeuroCult medium (Stem Cell Technology) containing 0.8% (w/v) hyaluronic acid (Lifecore Biomedical) at a density of 0.5×10⁶ cells/ml. Sodium alginate was dissolved in 0.9% (w/v) saline to reach a concentration of 1.5% (w/v) alginate and autoclaved. With an extruder (see e.g., FIGS. 1 and 2), 10 ml of cell solution and 10 ml of alginate solution were extruded into the 100 ml of sterile buffer containing 100 mM CaCl₂ at room temperature to form alginate hollow fibers with cells suspended in the hollow space. The fibers were crosslinked in the CaCl₂ solution for 10 minutes at room temperature. The CaCl₂ solution was removed and replaced with NeuroCult medium. Cells were cultured in the hollow fibers suspended in the medium in a regular cell culture incubator at 37° C., with 5% CO₂ and 95% air at 1 atm for 7 days. Single cells grew into aggregates. To harvest cells, the NeuroCult Medium was removed and replaced with PBS containing 40 mg/ml alginate lyase (Sigma-Aldrich) at 37° C. for 10 minutes. The alginate fibers were dissolved and cell aggregates were harvested. These aggregates can be dissociated into single cells by treating them with 0.05% trypsin (Life Technology) at 37° C. for 10 minutes. Cells can be processed into the alginate hollow fibers for a second round of expansion. The cells grew into large aggregates from single cells (see FIGS. 7A-7C).

### Example 4

In this Example, mouse L cells engineered to express Wnt 3A proteins were cultured for producing recombinant proteins in hollow fibers.

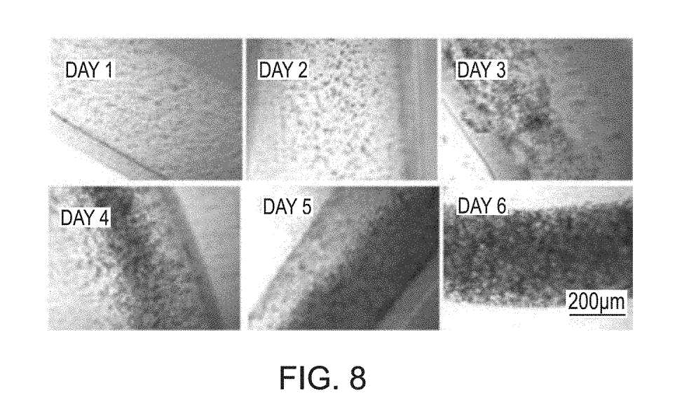

Mouse L cells stably expressing Wnt 3A proteins (ATCC® CRL-2647) were cultured in the hollow fibers for 20 days. Single cells were suspended in DMEM medium (Stem Cell Technology) containing 0.8% (w/v) hyaluronic acid (Lifecore Biomedical) at a density of 1×10⁶ cells/ml. Sodium alginate was dissolved in 0.9% (w/v) saline to reach a concentration of 1.2% (w/v) alginate and autoclaved. With an extruder (see FIGS. 1 and 2), 20 ml of cell solution and 20 ml of alginate solution were extruded into the 200 ml of sterile buffer containing 100 mM CaCl₂ at room temperature to form alginate hollow fibers with cells suspended in the hollow space. The fibers were crosslinked in the CaCl₂ solution for 10 minutes at room temperature. The CaCl₂ solution was removed and replaced with DMEM medium containing 10% FBS (Atlanta Biologicals). Cells were cultured in the hollow fibers suspended in the medium in a regular cell culture incubator at 37° C., with 5% CO₂ and 95% air at 1 atm for 20 days. Cells grew into high density aggregates by day 6 (see FIG. 8).

### Example 5

In this Example, various cells were suspended and grown in hollow alginate hydrogel fibers (also referred to as the cell culture system or culture system).

### Materials and Methods

Materials: Fib-iPSCs (iPSCs reprogrammed from human dermal fibroblasts) and MSC-iPSCs (iPSCs reprogrammed from human mesenchymal stem cells) were obtained from George Q. Daley laboratory (Children's Hospital Boston, Boston). H9 hESCs were purchased from WiCell Research Institute. L Wnt-3A cells (ATCC® CRL2647™) were acquired form ATCC. Reagents and their supplies: E8 medium (E8), Accutase and Live/Dead cell viability staining kit: Life Technologies; Y-27632: Selleckchem; Matrigel: D Biosciences; Sodium Hyaluronate (HA 700K-1): Lifecore Biomedical. Sodium alginates (500˜600 cp; 300˜400 cp and 80˜120 cp): Wako Chemicals. Sodium alginate (A2033-100G): Sigma. Vybrant cell-labeling solutions: Molecular Probes, Inc. DMEM: GE Healthcare Life Sciences; FBS: Atlanta biologicals; G418: Sigma. Antibodies and their supplies: Oct4 (Santa Cruz Biotechnology; 1:100); FOXA2 (Santa Cruz Biotechnology; 1:200); α-SMA (Abcam; 1:200); Nestin (Millipore; 1:200). Nanog (10 mg/mL), Oct4 (10 mg/mL), SSEA-4 (10 mg/mL) and alkaline phosphatase (10 mg/mL) and Brachyury (10 mg/mL) (R&D systems, Inc.). Syringe pump (New Era Pump System, Inc.); Disposable syringes (Henke sass wolf); Clear acrylic rectangular bar, steel tubes and plastics tubes (McMaster); Calcium chloride (Acros Orcanics); Sodium Chloride (Fisher scientific). Mechanical stage and controller (CESCO); Bellows bottles (Spectrum Chemical Mfg. Corp.); Luciferase assay kit (Biovision, K801-200).

Processing alginate hollow fibers: a home-made micro-extruder was used to process alginate hollow fibers. A hyaluronic acid (HA) solution containing single cells and an alginate solution was pumped into the central and side channel of the home-made micro-extruder, respectively, and extruded into a CaCl₂ buffer (100 mM) to make hollow fibers. Subsequently, CaCl₂ buffer was replaced by cell culture medium.

Culturing hPSCs in the hollow alginate hydrogel fibers: for a typical cell culture, 20 μL cell solution in alginate hollow fibers were suspended in 2 mL E8 medium in a 6-well plate and cultured in an incubator with 5% CO₂, 21% O₂ at 37° C. Medium was changed daily. To passage cells, medium was removed and alginate hydrogels were dissolved with 0.5 mM EDTA for 5 minutes. Cell mass was collected by centrifuging at 100 g for 5 minutes, treated with Accutase at 37° C. for 12 minutes and dissociated into single cells for following culture.

Culturing L-Wnt3A-cells in the hollow alginate hydrogel fibers: for a typical cell culture, 20 μL cell solution in alginate hollow fibers were suspended in 2 mL DMEM medium plus 10% FBS and 0.4 mg/mL G418 in a 6-well plate and cultured in an incubator with 5% CO₂, 21% O₂ at 37° C. Medium was changed daily and collected for quantifying Wnt3A proteins. To quantify Wnt3A proteins, MDA-468 cells (ATCC® HTB-132™) were stably transfected with a luciferase reporter for the canonical Wnt signaling (Addgene, #24308). These MDA-468-TFP cells were plated in a 96-well plate (5000 cells/well/200 mL medium). 24 hours later, 150 mL fresh DMEM plus 10% FBS and 50 mL L-Wnt3A-cells conditioned medium was added and incubated for another 18 hours. Medium was then removed and cells were washed with PBS once before 200 mL cell lysis buffer was added and incubated for 10 minutes at room temperature. 50 mL cell lysates, 50 mL substrate A and 50 mL substrate B from the luciferase assay kit were mixed and the light signals were immediately read with a luminometer. The quantity of Wnt3a protein was calculated with a standard curve.

Culturing hPSCs in the hollow alginate hydrogel fibers with bioreactors: 2.0 mL cell solution in hollow fibers was suspended in a home-made bioreactor. Cells were cultured in an incubator with 5% CO₂, 21% O₂ at 37° C. for 10 days. For bioreactor 1, medium was stored in a flask and continuously perfused into the bioreactor. For bioreactor 2, medium was stored in a bellow that was periodically pressed to flow the medium into or released to withdraw the medium from the container.

Staining and imaging: Cells cultured on 2D surfaces were fixed with 4% paraformaldehyde (PFA) at room temperature for 15 minutes, permeabilized with 0.25% Triton X-100 for 15 minutes, and blocked with 5% donkey serum for 1 hour. Cells were then incubated with primary antibodies at 4° C. overnight. After extensive washing, secondary antibodies and 4′,6-Diamidino-2-Phenylindole, Dihydrochloride (DAPI) were added and incubated for another 1 hour at room temperature. Cells were washed with PBS for 3 times before imaging with a Zeiss Axio Observer Fluorescent Microscopy. To assess the pluripotency of cells, hPSCs were plated onto the Matrigel-coated plate overnight before fixation and staining. The percentage of Oct4+ or Nanog+ nuclei was quantified with Image J software. At least 1000 nuclei were analyzed. To stain 3D cylindrical cell mass, the cell mass was harvested and fixed with 4% PFA at room temperature for 30 minutes, then incubated with PBS+0.25% Triton X-100+5% goat serum+primary antibodies at 4° C. for 48 hours. After extensive washing, secondary antibodies in 2% BSA were added and incubated at 4° C. for 24 hours. Cells were washed with PBS for 3 times before imaging with Nikon A1 Confocal Microscopy. LIVE/DEAD® Cell Viability staining was used to assess live and dead cells, according to the product manual.

Embryoid body (EB) differentiation: hPSCs released from the hollow alginate hydrogel fibers were suspended in DMEM+20% FBS+10 μM β-mercaptoethanol in a low adhesion plate for 6 days. The cell mass was then transferred onto plates coated with 0.1% gelatin and cultured in the same medium for another 6 days, followed by fixation and staining as above.

Teratoma formation in vivo: all animal protocols were approved by the Institutional Animal Care and Use Committee of the University of Nebraska-Lincoln. All experimental procedures involving animals were performed in accordance with the guidelines of the Institutional Animal Care and Use Committee of the University of Nebraska-Lincoln. 2×10⁶ hPSCs were suspended in 25 μL PBS plus 25 μL Matrigel and injected subcutaneously at the back of the neck of the NOD-SCID mice (Charles River Laboratory). Tumors were harvested after 6-12 weeks. The tumors were fixed with 4% PFA for 48 hours and sequentially dehydrated with 70%, 95%, and 100% ethanol, and defatted with xylene for 2 hours before embedding in paraffin. Then 10 μm thick sections were cut and stained with hematoxylin and eosin.

Karyotype: Karyotyping was performed by WiCell Research Institute.

Mesodermal induction: H9 cells in hollow fibers were cultured in E8 medium for 7 days, then in DMEM/F12 medium with 1% B27 minus insulin and 12 mM CHIR99021 for 24 hours before fixation and staining.

Endodermal induction: H9 cells in hollow fibers were cultured in E8 medium for 7 days, then in RPMI 1640 medium with 1% GlutaMAX, 1% B27 minus insulin, 4 mM CHIR99021 for 24 hours and in RPMI 1640 medium with 1% GlutaMAX, 1% B27 minus insulin for additional 24 hours before fixation and staining.

Cardiomyocyte differentiation: H9 cells in hollow fibers were cultured in E8 medium for 7 days, then in DMEM/F12 with 1% B27-insulin between for 6 days, and DMEM/F12 with 1% B27 for 9 days. The following small molecules were added during the differentiation: 12 mM CHIR99021 for days 0-1; 5 mM IWR1 for days 3-4. Cell mass were released on day 11 to gelatin coated plate. Beating cardiomyocytes were filmed on day 15. Some samples were fixed on day 11 for cTNT immunostaining.

Statistical analysis: Statistical analyses were done using the statistical package Instat (GraphPad Software, La Jolla, Calif.).

### Results

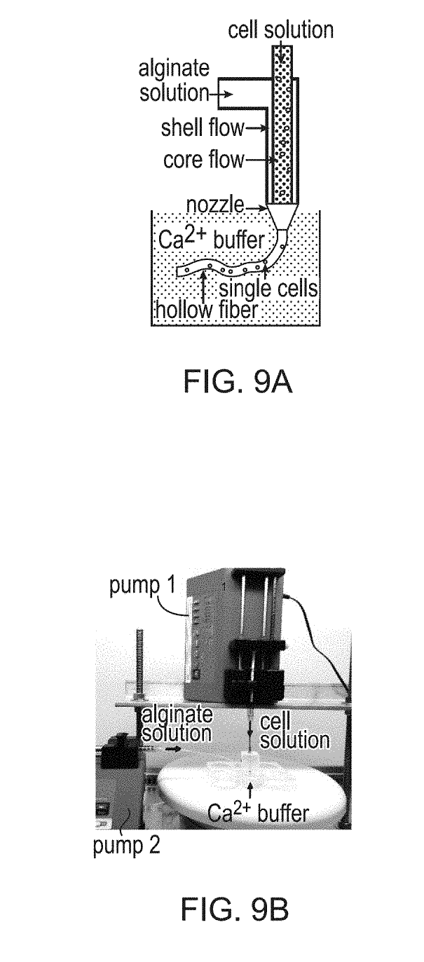

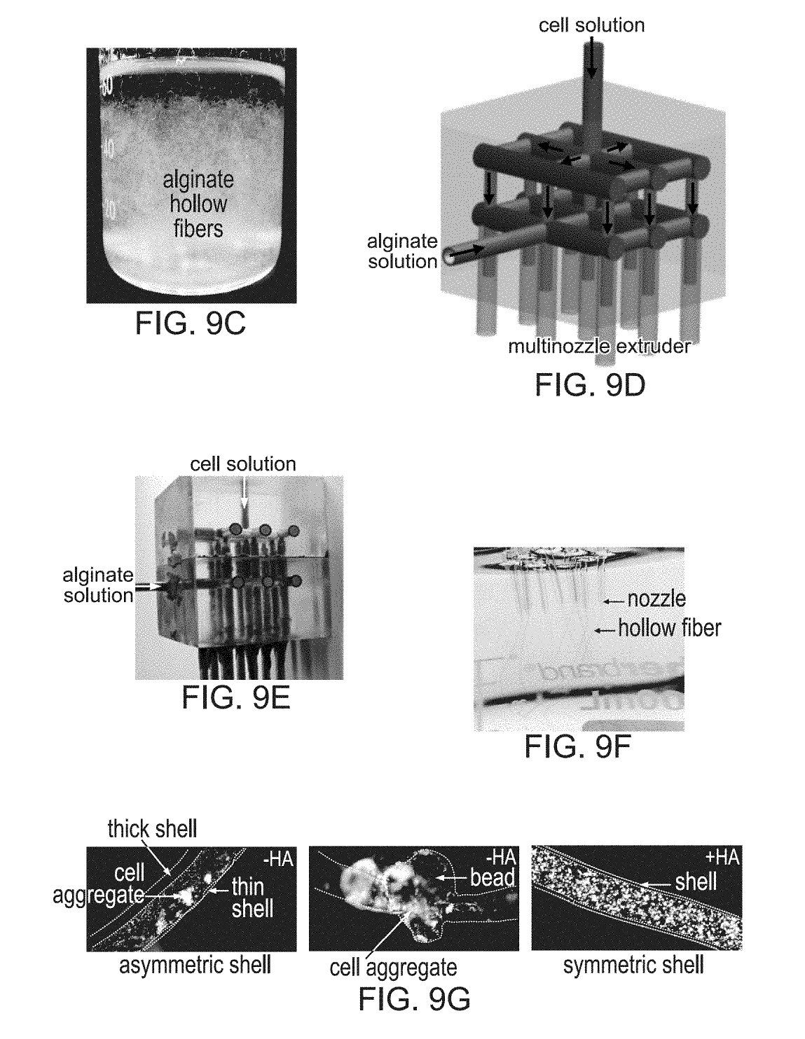

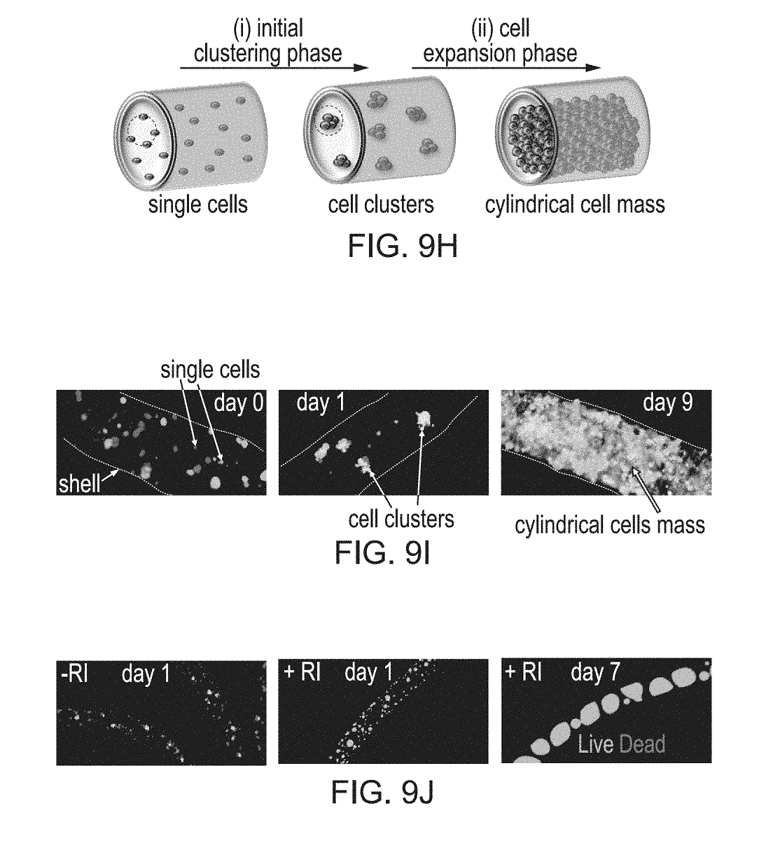

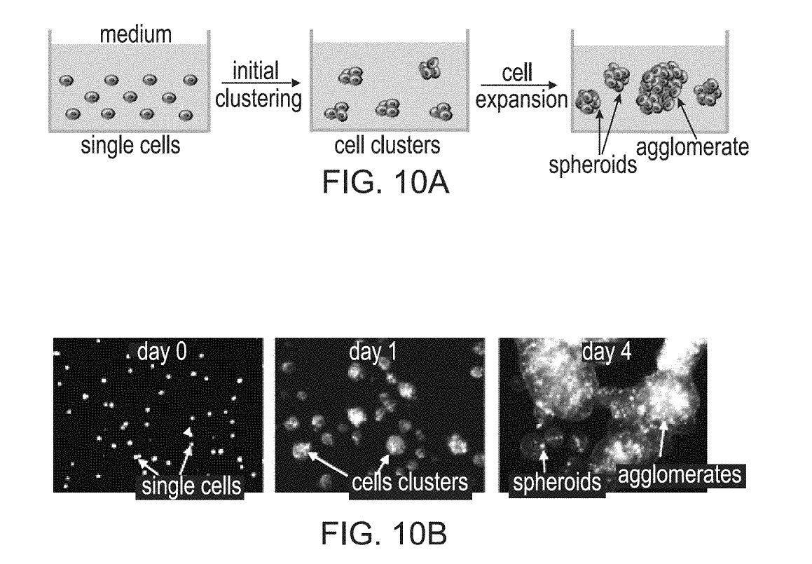

A micro-extruder was made for processing hollow fibers with alginate hydrogels (FIGS. 9A & 9B). The extruder could have one or multiple nozzles for simultaneously processing one or multiple hollow fibers (FIGS. 9A-9F). It was found that the viscosity of the cell solution and alginate solution should be close to process defect-free hollow fibers. Both hyaluronic acid (HA) and methylcellulose (MC) solutions could be used to suspend the cells for this purpose. Without HAs or MCs, hollow fibers with asymmetric shells or beads were frequently formed (FIG. 9G). Both defects could lead to cell culture failure. Similar to hPSCs in suspension cultures (FIGS. 10A & 10B). hPSCs in the hollow fiber cell culture system grew through an initial clustering phase and a subsequent cell expansion phase (FIGS. 9H & 9I) and the ROCK inhibitor Y-27632 was required for the initial survival of the dissociated hPSCs (FIG. 9J).

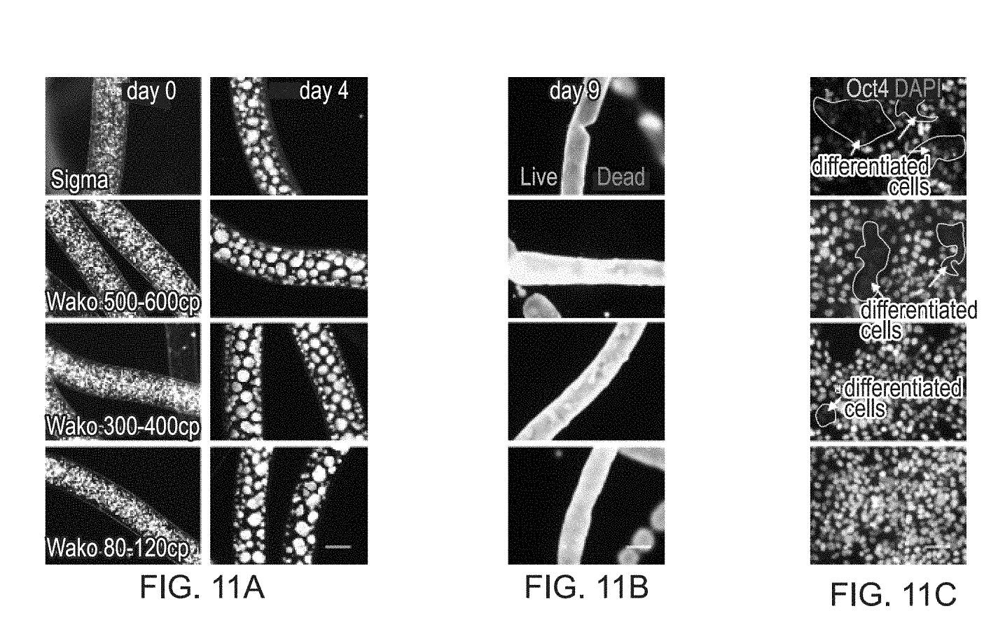

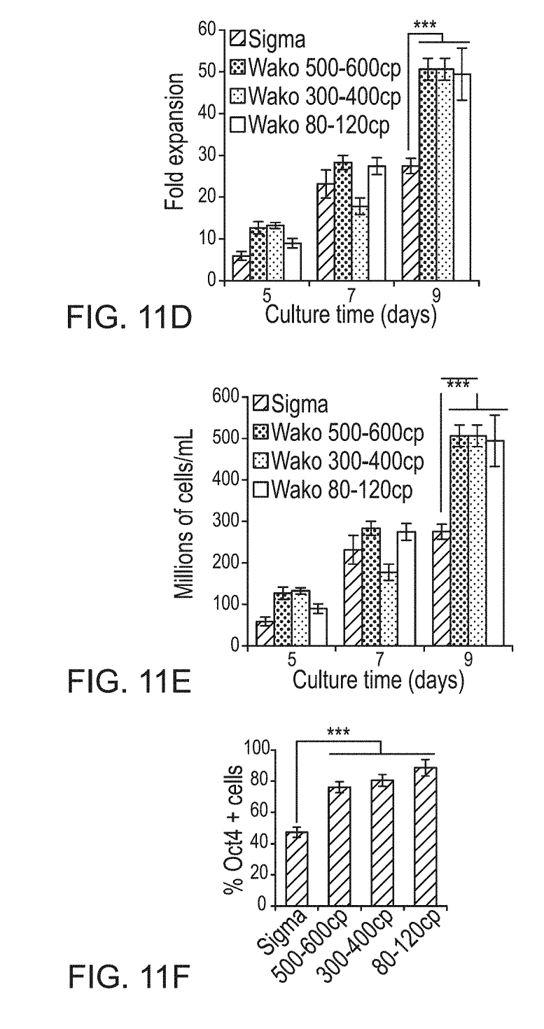

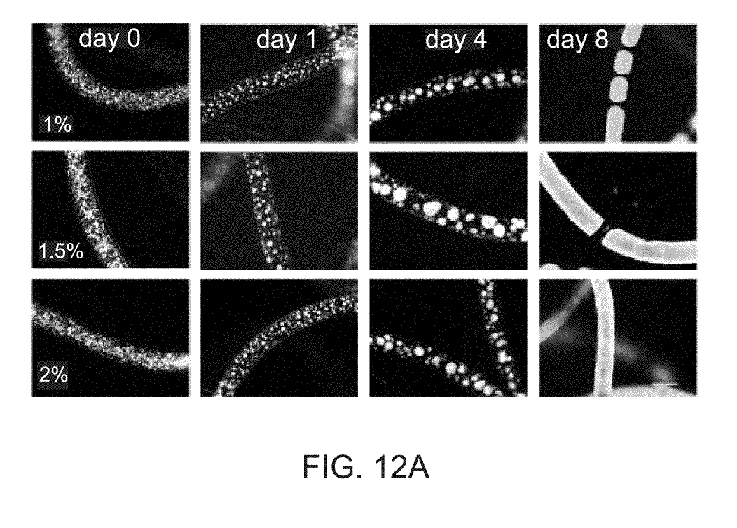

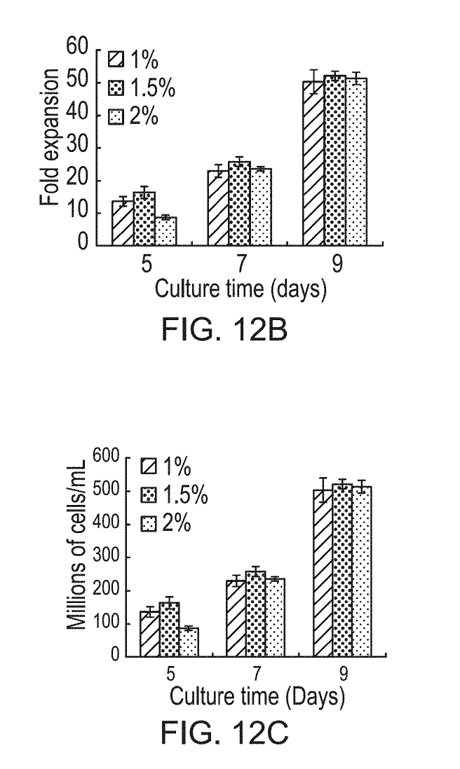

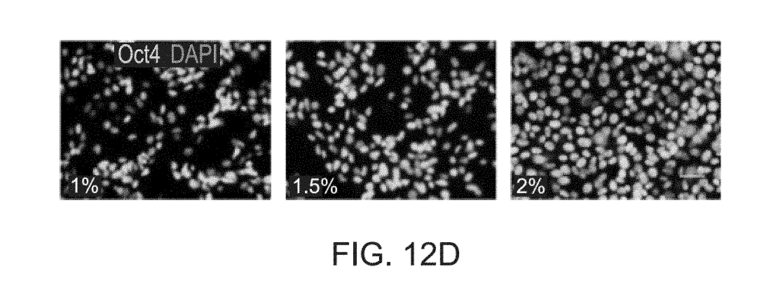

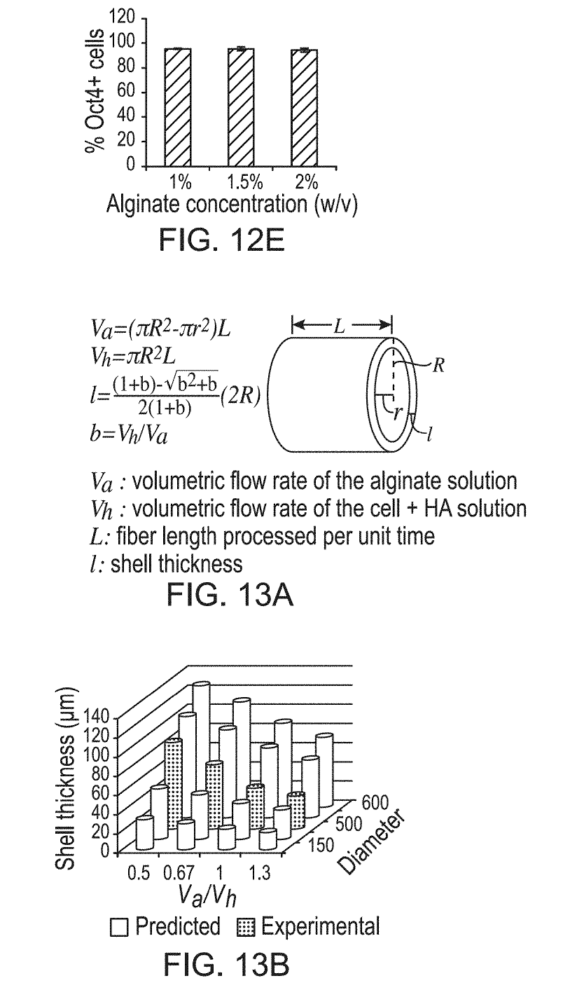

It was found that the proliferation and pluripotency of hPSCs in the fibers were significantly influenced by the alginate hydrogel formulation. For instance, when cultured in hollow fibers processed from 2% alginates from Sigma (#A2033-100G) and Wako Chemicals (500˜600 cp; 300˜400 cp and 80˜120 cp) for 9 days, hPSCs expanded 27-, 51-, 51- and 49-fold to yield 2.7×, 5.1×, 5.1× and 4.9×10⁸ cells/mL with 47%, 76%, 80% and 89% of the final cells expressing the pluripotency marker Oct4, respectively (FIGS. 11A-11F). Live/dead cell staining revealed almost no cell death for all the cultures (FIG. 11B). Compared with the alginate type, the influence of alginate concentration in the range of 1.0% to 2.0% was much less. For instance, there was no significant difference in cell proliferation and pluripotency for hPSCs cultured for 9 days in hollow fibers processed with 1.0%, 1.5% and 2.0% Wako Chemicals 80-120 cp alginates (FIGS. 12A-12E). It was concluded that 1.5% Wako Chemicals 80-120 cp alginate hydrogel was appropriate for culturing hPSCs in the hollow alginate hydrogel fiber cell culture system.

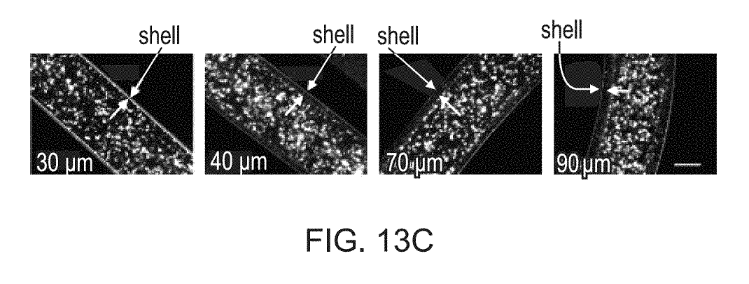

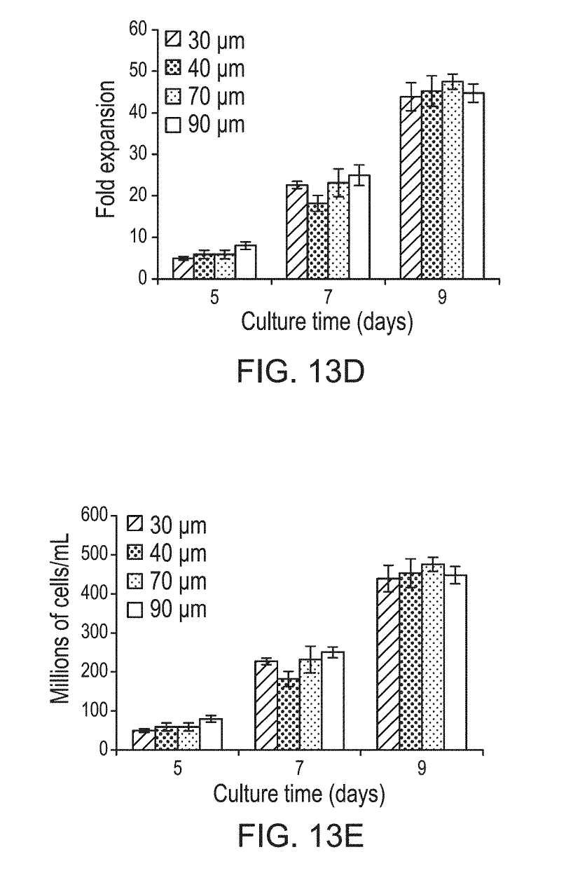

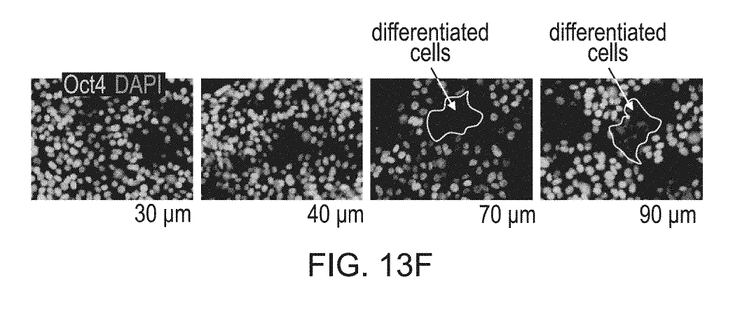

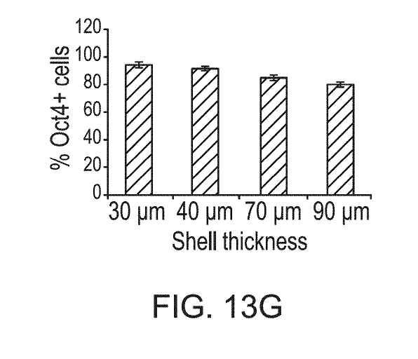

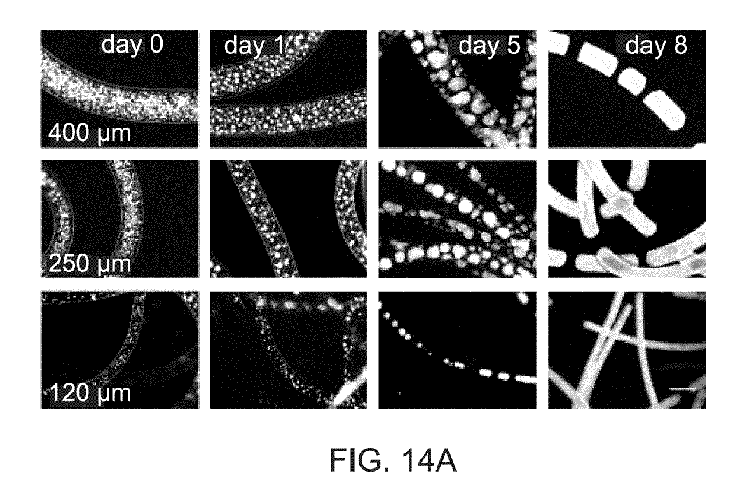

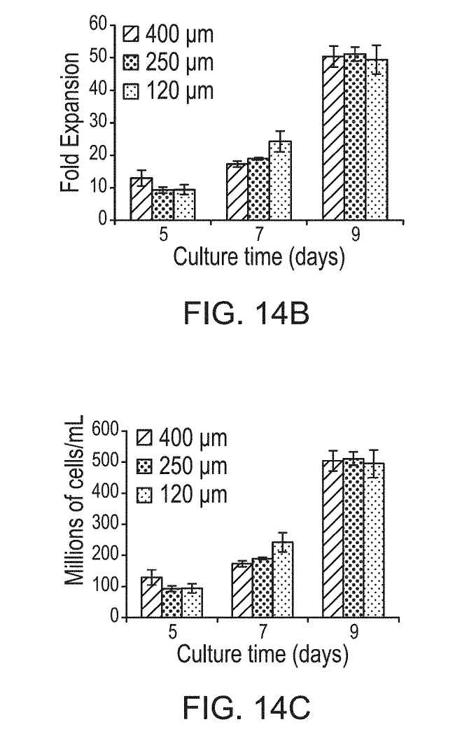

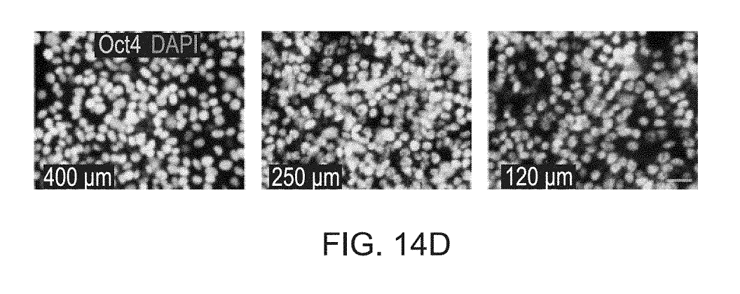

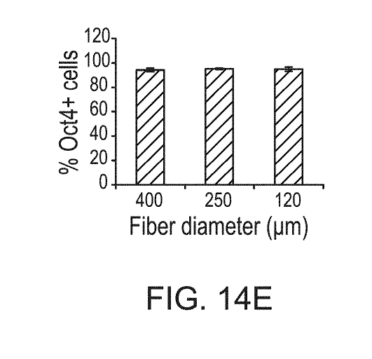

The fiber geometry also influenced hPSC culture in the cell culture system. At a given fiber outer diameter, the hydrogel shell thickness can be controlled by varying the ratio of the cell solution and alginate solution flow rate and can be predicted with the Equation described in FIGS. 13A & 13B. The fiber outer diameter is roughly equal to the inner diameter of the extruder nozzle. When cultured in hollow fibers with 30, 40, 70 and 90 μm shells for 9 days, 94%, 92%, 85% and 80% of the final cells retained the pluripotency marker Oct4. There was no significant difference in cell viability and expansion between the different conditions (FIGS. 13C-13E). When cultured in hollow fibers with inner diameter of 400 μm, 250 μm and 120 μm for 9 days, 95% of the cells retained the Oct4 marker (FIGS. 14-14E). It was concluded that hollow fibers with shell thicknesses<70 μm and inner diameters<400 μm were appropriate for culturing hPSCs in the hollow alginate hydrogel fiber cell culture system.

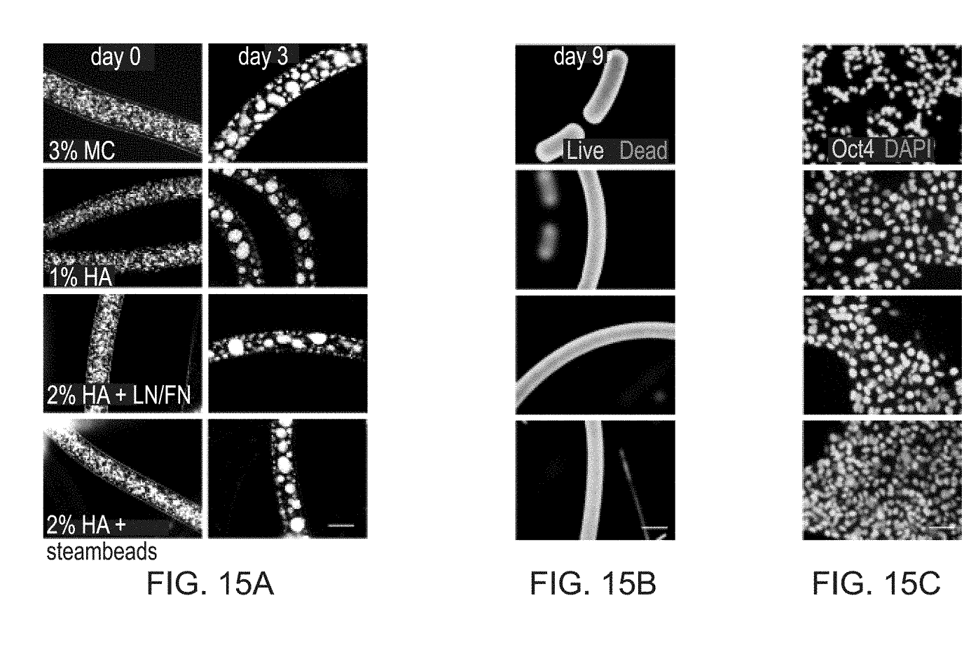

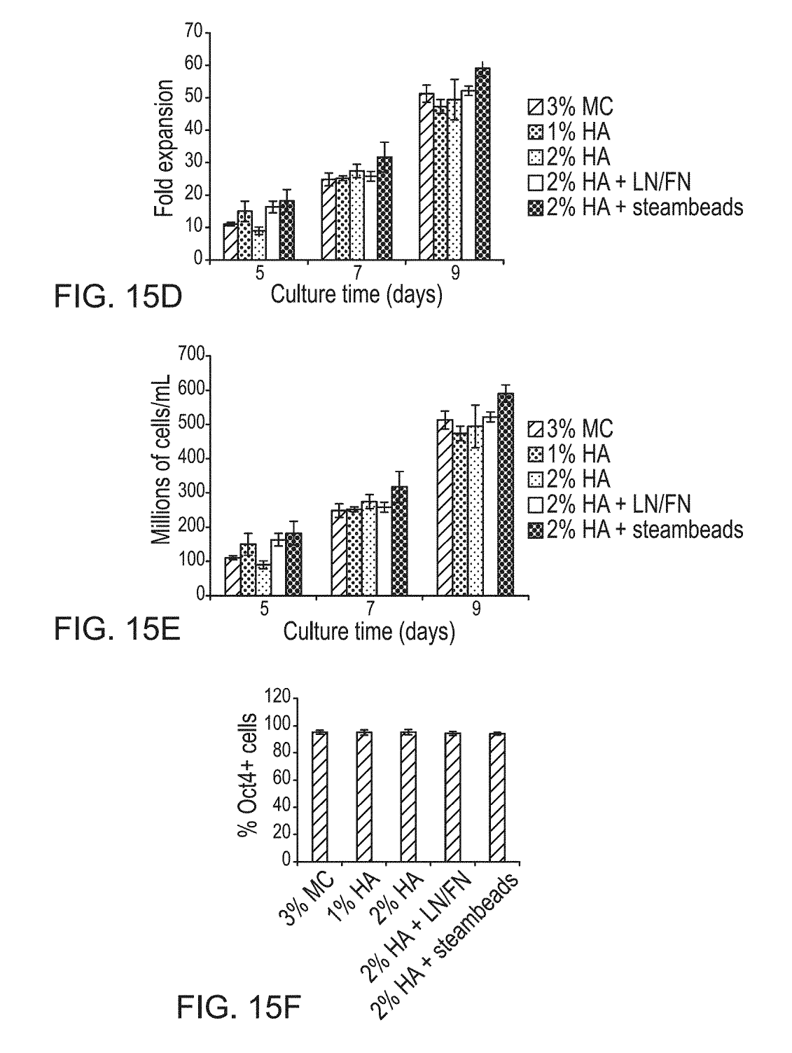

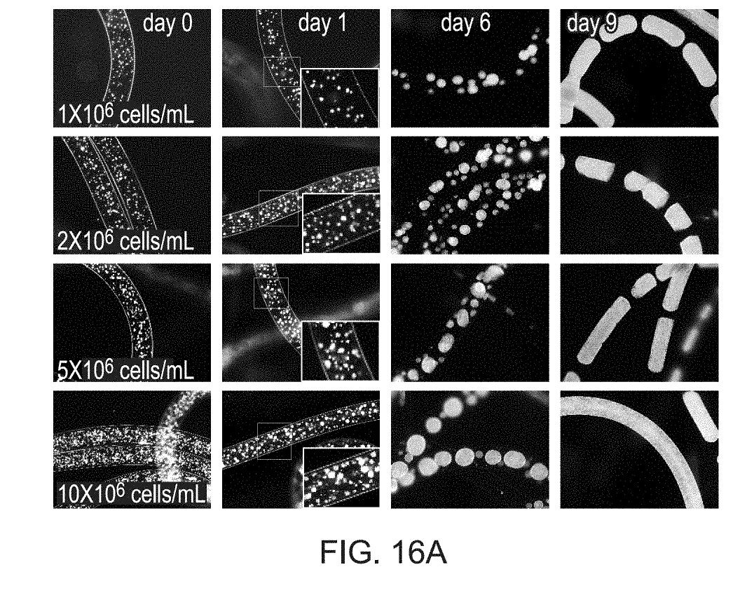

Research showed adding extracellular matrix proteins such as fibronectins and laminins enhanced hPSC culture efficiency in suspension cultures. The results of the instant Example showed these proteins at the tested concentrations did not improve the cell viability, growth rate and pluripotency and were unnecessary with the cell culture system (FIGS. 15A-15F). Since both HAs and MCs could be used to cells, it was further analyzed whether they differentially influenced the cell culture. The results showed 1% HAs, 2% HAs and 3% MCs resulted in similar cell viability, expansion and pluripotency (FIGS. 15A-15F). A main concern with culturing hPSCs in alginate hollow fibers is that the large protein factors (e.g. bFGFs, insulins and transferrins) in the medium might not efficiently travel through the hydrogel shell and cell mass to feed the cells. When Poly Lactic-co-Glycolic Acid (PLGA) microspheres (StemBeads) containing and slowly releasing bFGFs were added to the liquid core of the hollow fibers, the cell viability, expansion and pluripotency were not improved, indicating the transport of proteins in the new culture system was efficient and sufficient (FIGS. 15A-15F).

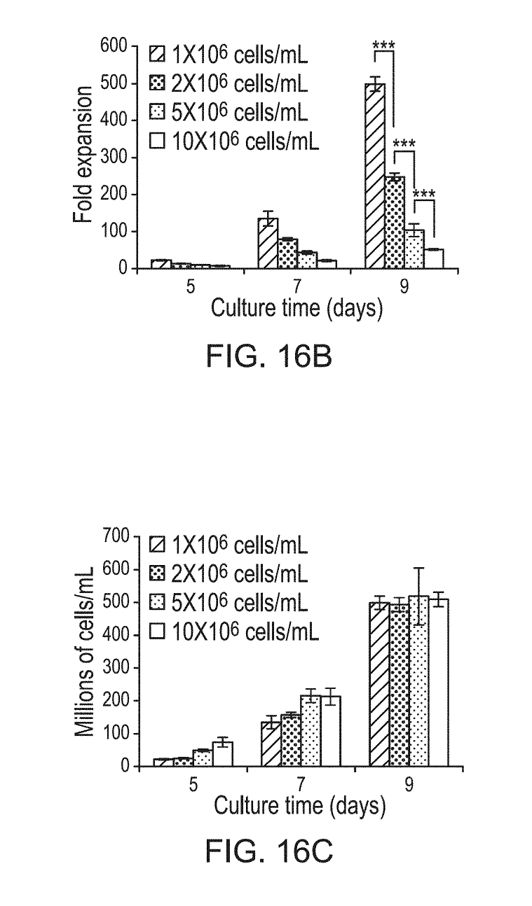

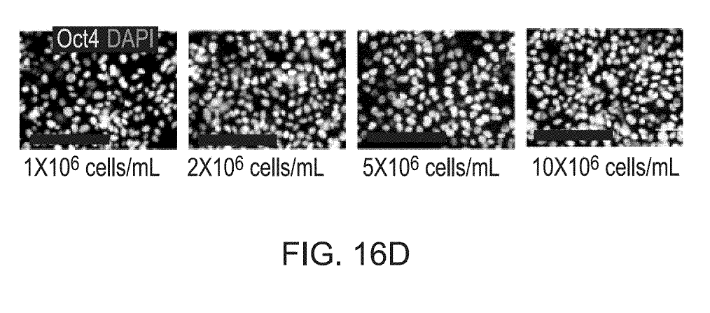

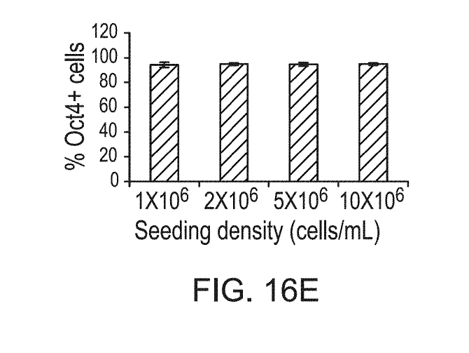

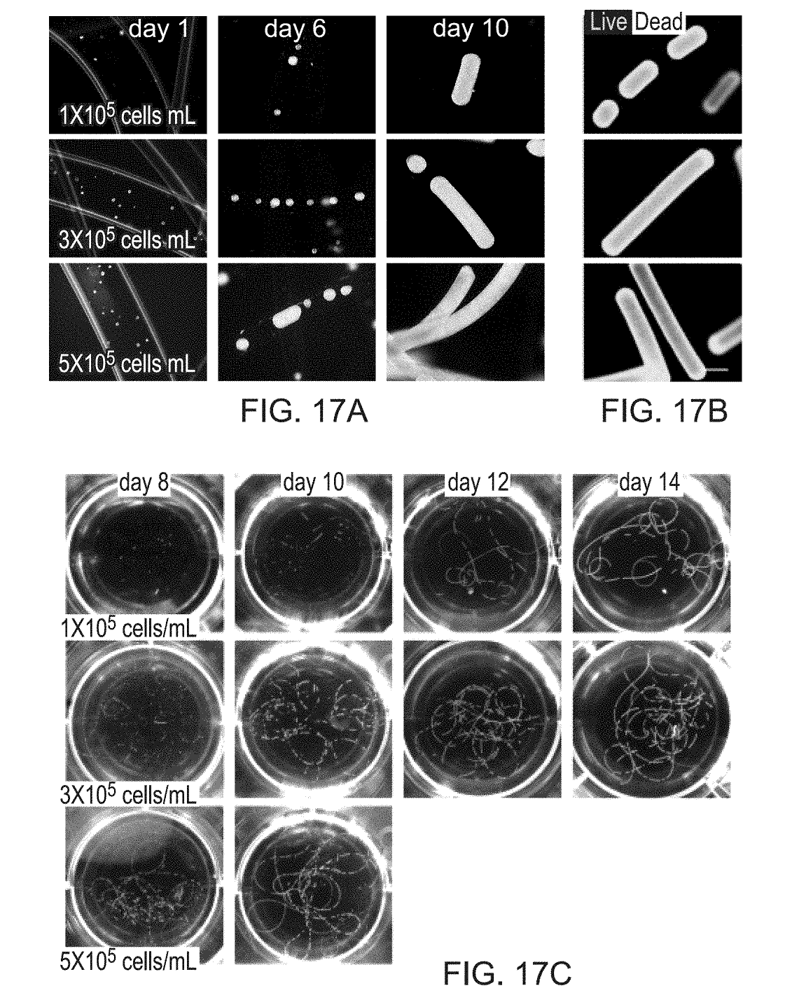

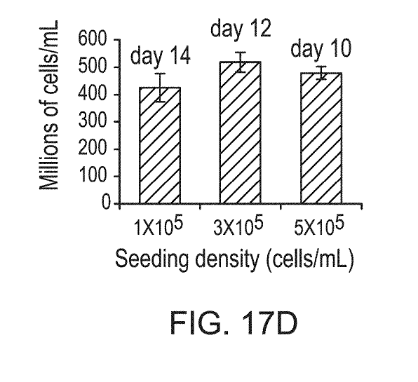

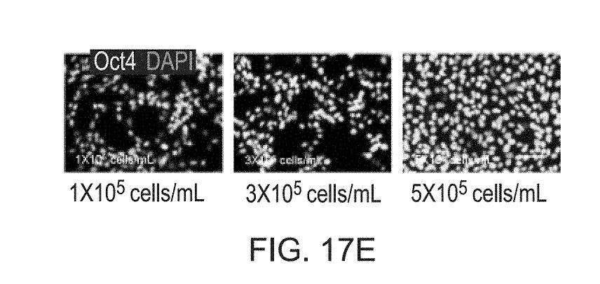

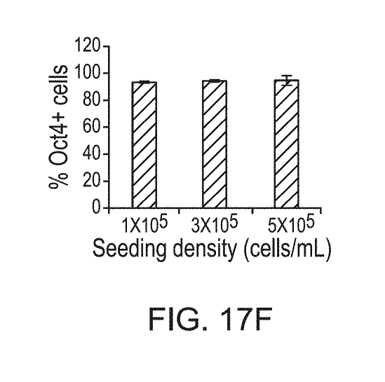

The influence of cell seeding density on hPSC culture in the cell culture system was also investigated. When seeded at 1.0×10⁶, 2.0×10⁶, 5.0×10⁶, 10.0×10⁶ cells/mL, hPSCs expanded 433-, 196-, 104- and 46-fold on day 9, respectively, yielding around 5.0×10⁸ cells/mL (FIG. 16B). For all conditions, cells grew through the aforementioned two phases (FIG. 16A). At 24 hours, the cell cluster size was larger for higher seeding density, but the number of cell cluster per volume was not significantly affected by the seeding density (FIG. 16A, day 1, insert). These results showed hPSCs grew faster at lower seeding density. However, the seeding density did not influence the pluripotency (FIGS. 16D & 16E). It was extremely exciting that hPSCs seeded at ultralow densities could grow as well without sacrificing cell viability and pluripotency. When seeded at 1.0×, 3.0× and 5.0×10⁵ cells/mL, hPSCs expanded 4000-, 1666- and 1000-fold to yield ˜4.2×, 5.1×, 4.8×10⁸ cells/mL on day 14, 12 and 10 respectively (FIG. 17D).

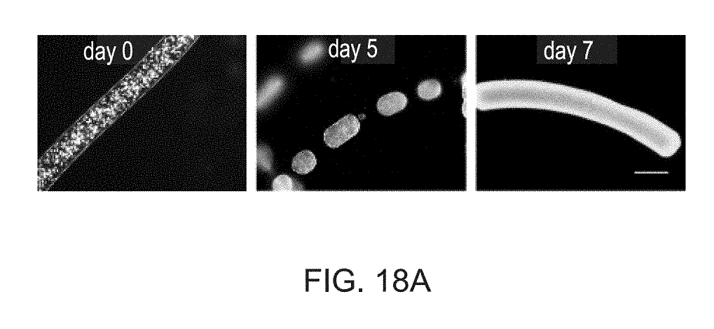

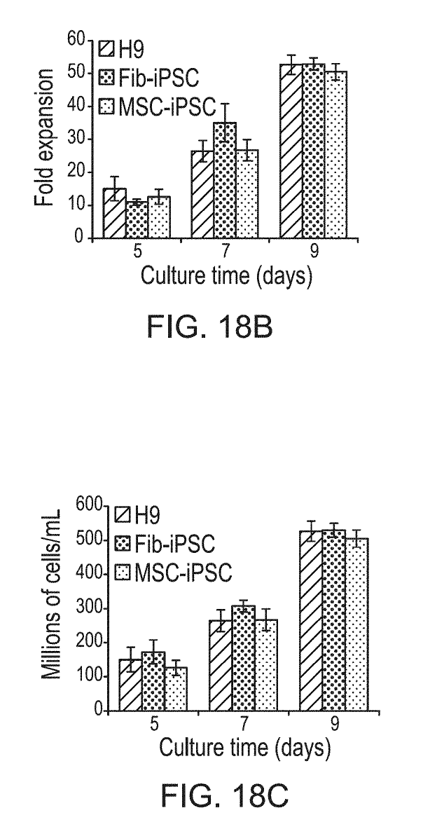

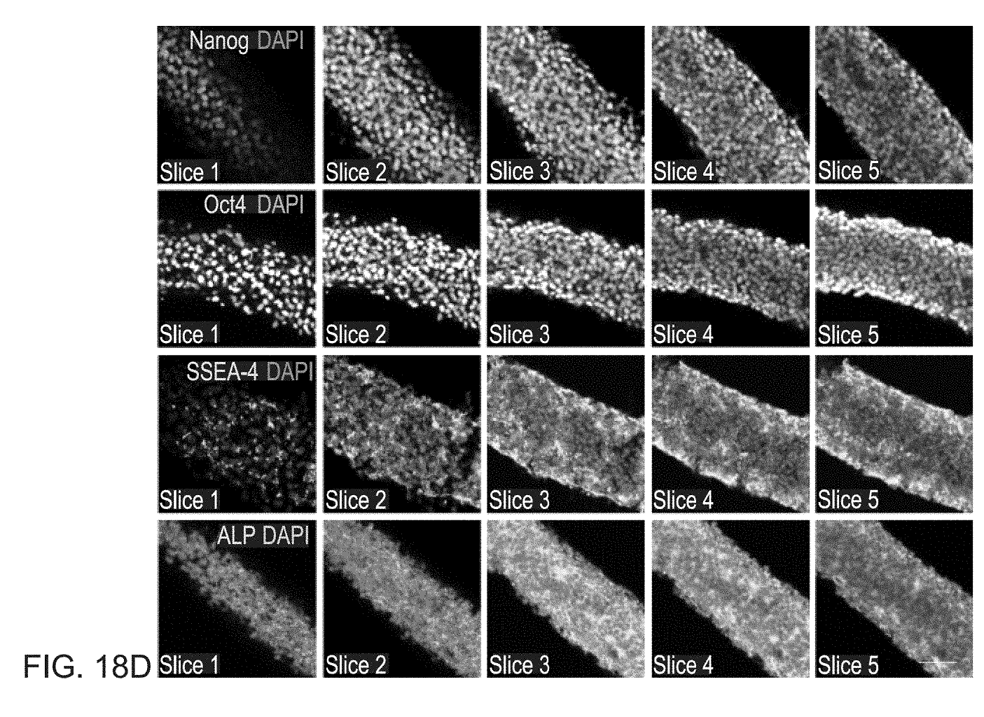

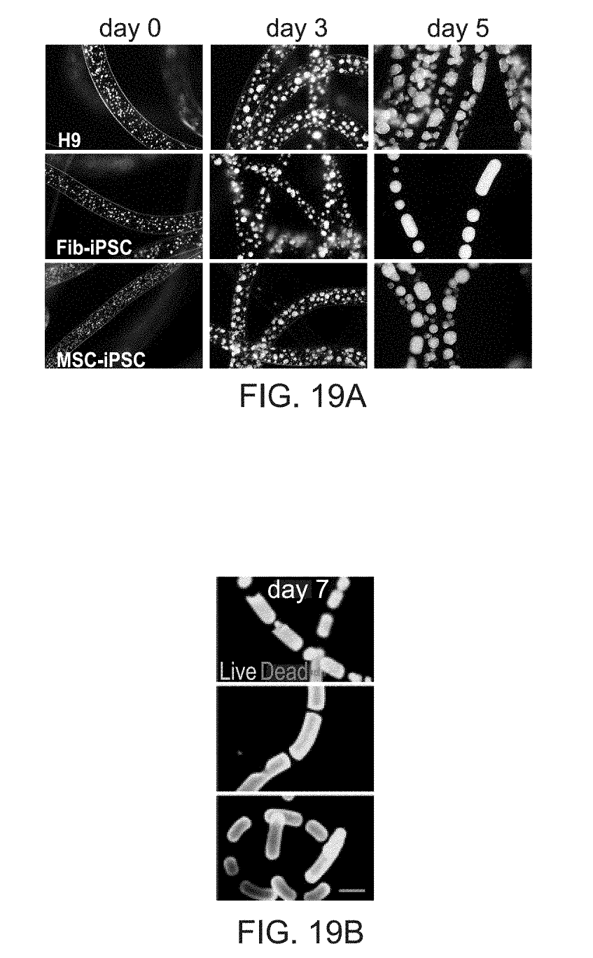

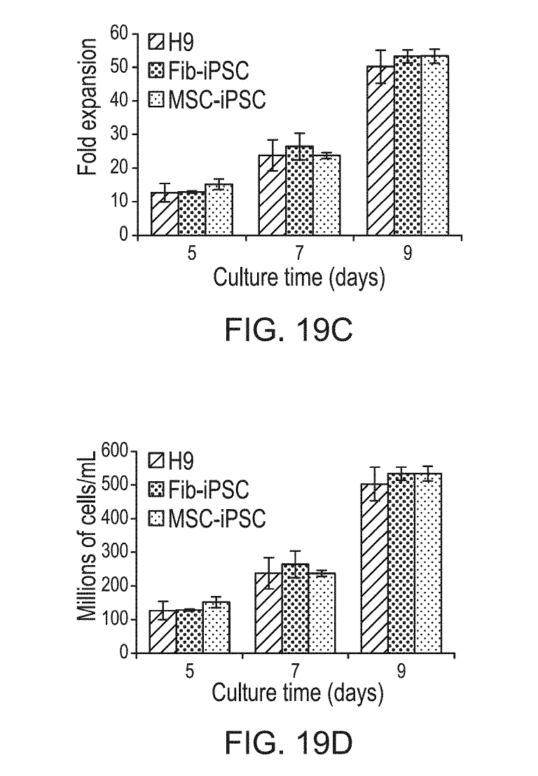

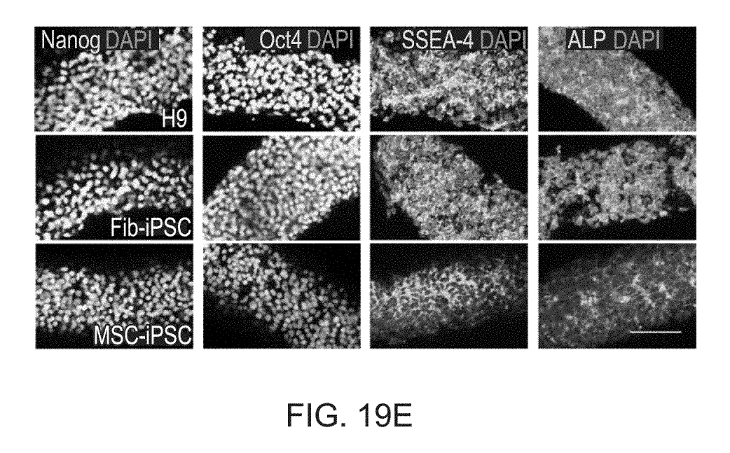

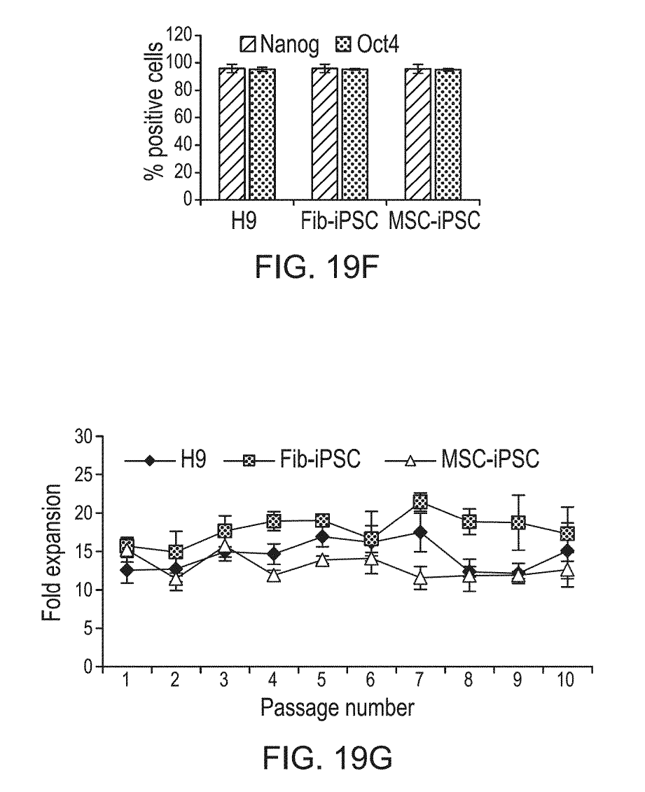

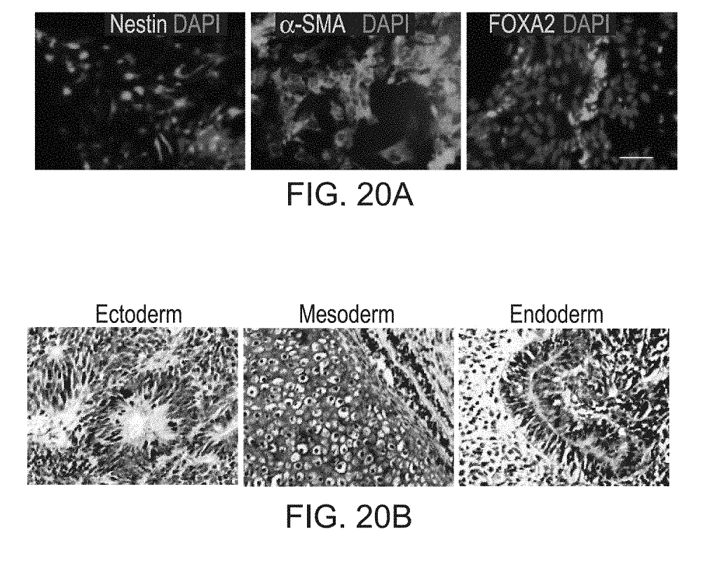

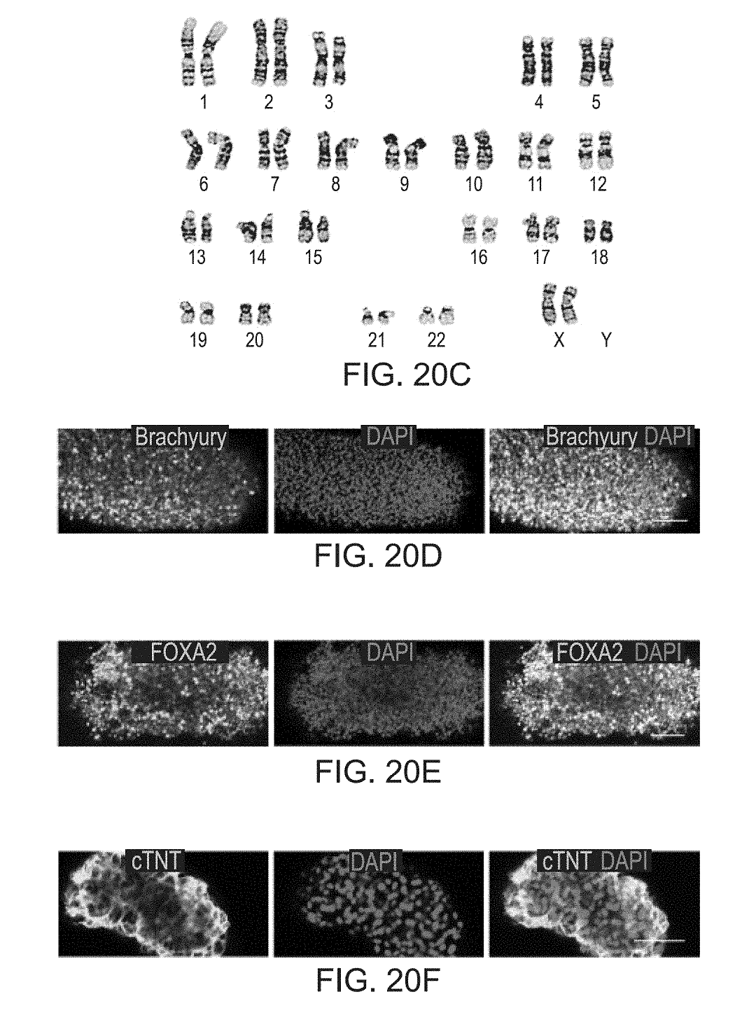

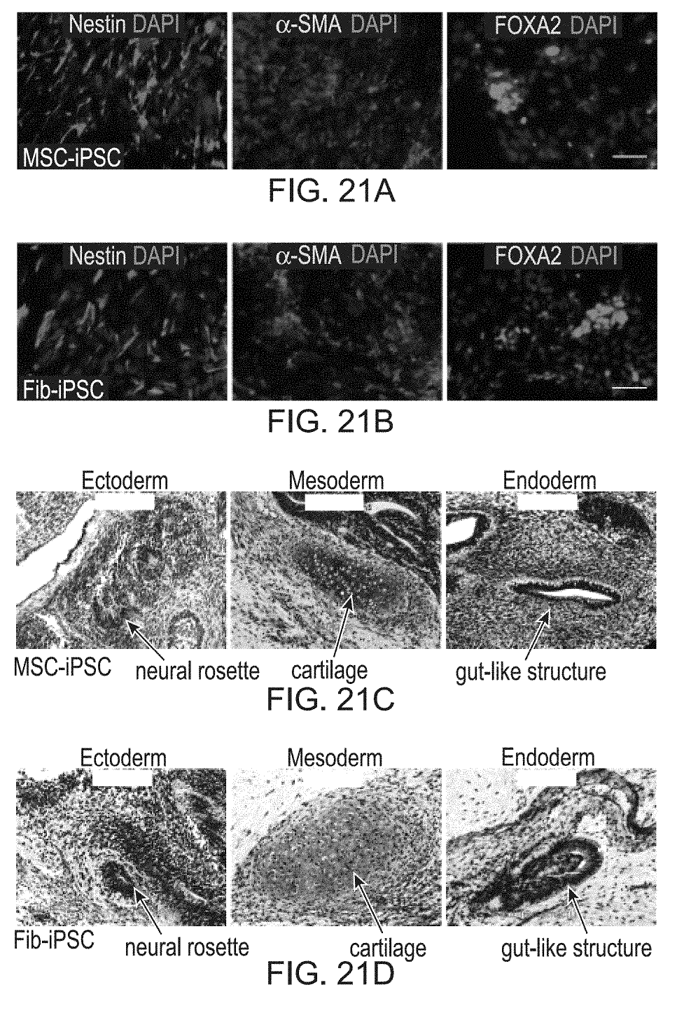

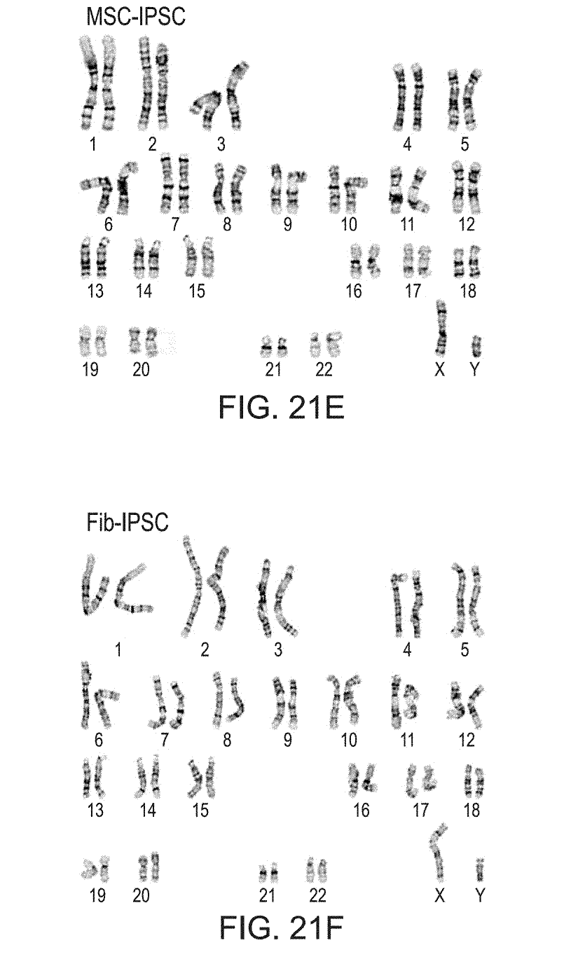

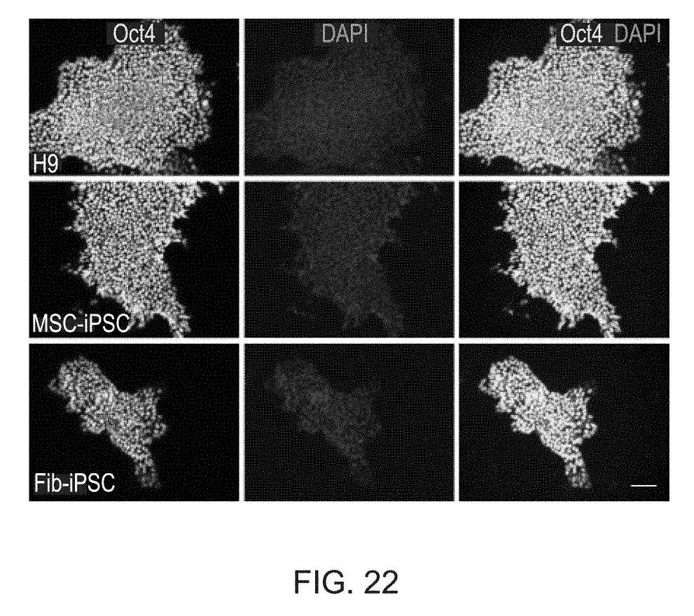

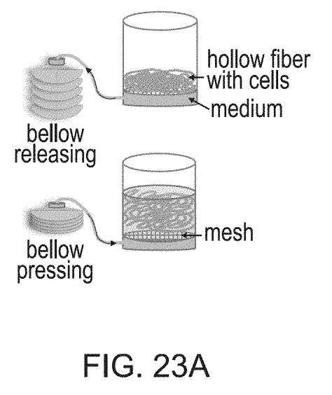

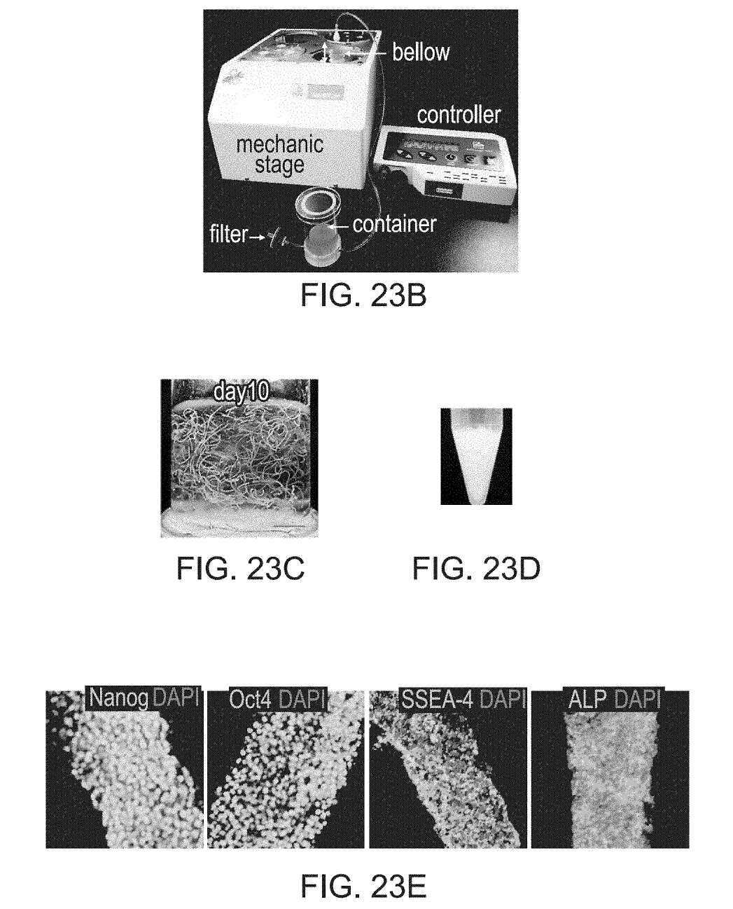

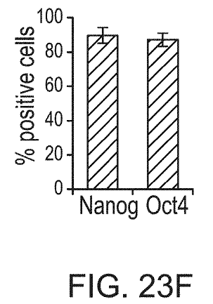

After optimization, the cell culture system was evaluated for culturing multiple hPSC lines for long term. All hPSCs grew well in the cell culture system and there were no significant difference in cell morphology, viability, growth rate and pluripotency between the hPSC lines (FIGS. 18A-18D). During a 10-passage culture in the cell culture system, when seeded at 1.0×10⁷ cells/mL, hPSCs consistently expanded ˜15-fold per passage per 5 days and >95% of the cells expressed Oct4 (FIG. 19G). The long-term culture in the cell culture system did not alter the cell phenotype as shown by the similar morphology, viability, growth kinetics and pluripotency to hPSCs at passage 1 (FIGS. 18A-18D and FIGS. 19A-19G). In vitro embryoid body (EB) differentiation and in vivo teratoma formation confirmed their pluripotency after the long-term culture. All hPSCs were successfully differentiated into FOXA2+ endodermal, α-SMA+ mesodermal and Nestin+ ectodermal cells in the EB assay (FIGS. 20A and 21A & 21B). All hPSCs formed teratomas containing endodermal, mesodermal and ectodermal tissues when transplanted to immune-deficient mice (FIGS. 20B and 21C & 21D). In addition, after the long-term culture, all hPSCs retained normal karyotypes (FIGS. 20C and 21E & 21F) and could be cryopreserved or further cultured on Matrigel-coated 2D surface (FIG. 22). After expansion and further culturing in a mesodermal or endodermal or a cardiomyocyte differentiation medium, hPSCs in the hollow fibers could be differentiated to the corresponding mesodermal cells or endodermal cells or cardiomyocytes at high efficiency, indicating the cell culture system supported hPSC differentiation (FIGS. 23D-23F).

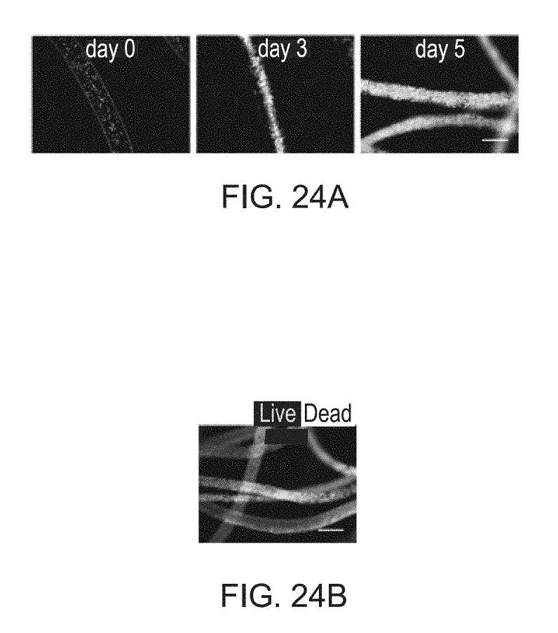

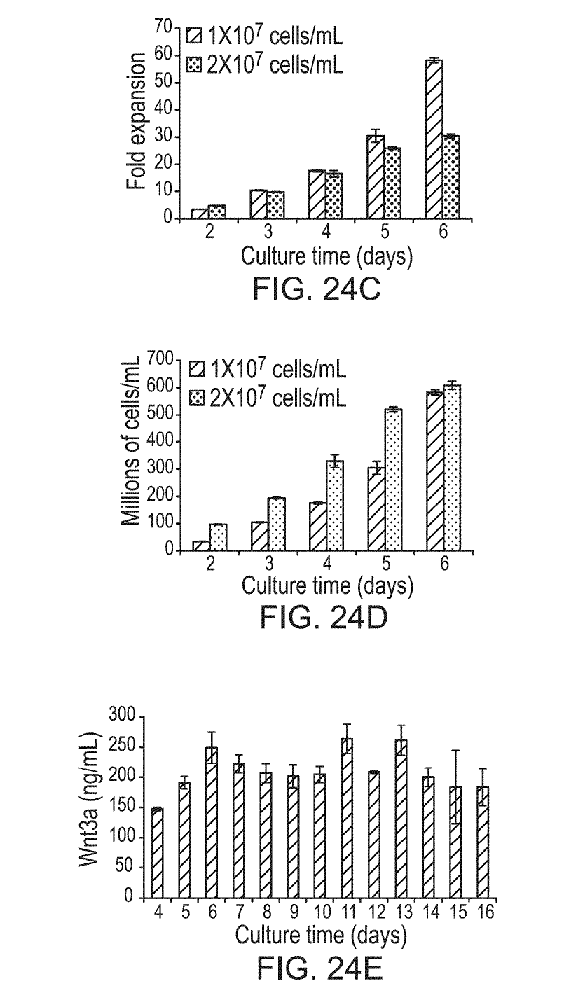

The culture system could be used to culture cells other than hPSCs. For instance, the murine L cells engineered to express Wnt3a proteins could be efficiently cultured without notable cell death, yielding around 6.0×10⁸ cells/mL. Importantly, these cells consistently expressed Wnt3a proteins during a 16-day culture at level similar to this expressed by L cells cultured in 2D dishes (FIGS. 24A-24E). This results demonstrated the potential of the hollow hydrogel fibers as a generally applicable system for culturing cells.

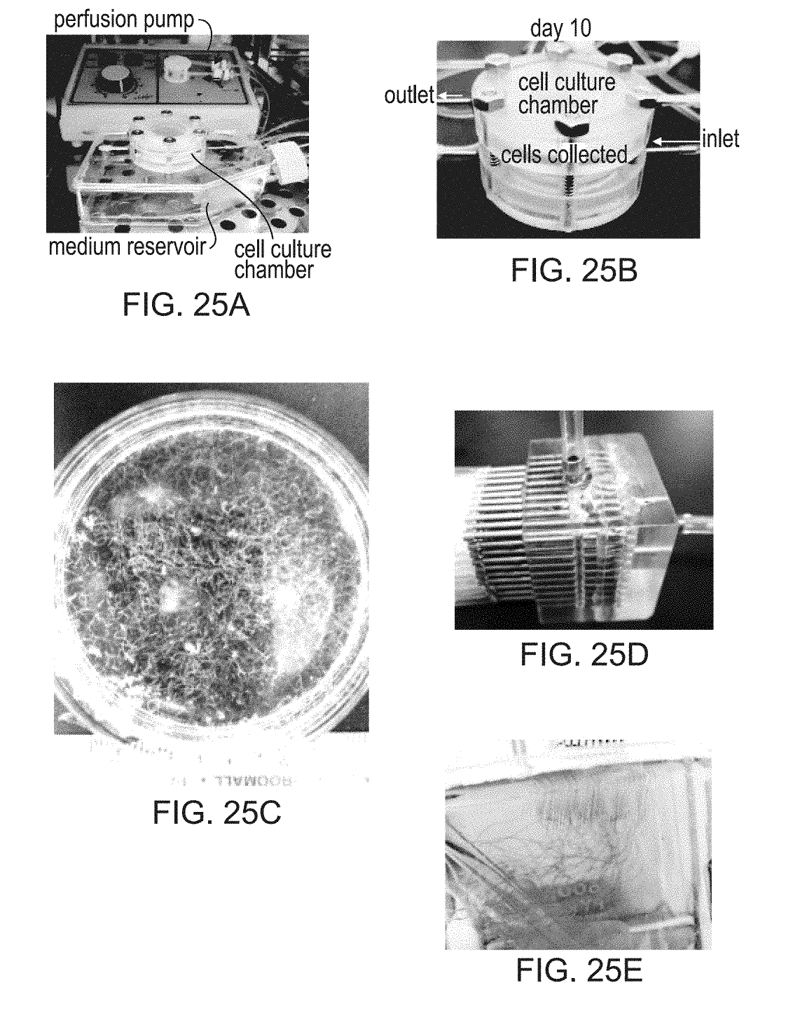

Two prototype bioreactors were designed and built for the cell culture system. Hollow fibers with cells were processed into a cylindrical container. In Bioreactor 1, medium stored in a flask was continuously perfused into the container (FIGS. 25A-25C). In Bioreactor 2, medium was stored in a plastic bellow that could be pressed to flow the medium into or released to withdraw the medium from the container, respectively (FIGS. 23A-23F). hPSCs in both bioreactors grew well and yielded ˜5.0×10⁸ cell/mL on day 10. >95% of cells expressed the pluripotency markers. These prototype bioreactors could be scaled up in the future. To scale up the processing of hollow alginate hydrogel fibers, an extruder was also made with 100 nozzles that could process 1 liter hollow fibers within 30 minutes (FIGS. 25D & 25E).

These results demonstrated that the methods and devices of the present disclosure can be used to culture and manufacture cells in hollow alginate hydrogel fibers. It is contemplated that the methods may be useful in both research laboratories and industry for preparing sufficient and high quality cells for disease and injury treatments, screening libraries for drugs, and manufacturing proteins and vaccines.

What is claimed is:

1. A method of manufacturing cells at various scales, the method comprising:
suspending a cell solution including cells in the hollow space of alginate hydrogel fibers;
suspending the fibers including the cells in cell culture medium; and
culturing the cells.

2. The method of claim 1 wherein the alginate hydrogel fibers comprise alginate polymers selected from the group consisting of alginate acid polymers, sodium alginate polymers, modified alginate polymers, and combinations thereof.

3. The method of claim 1 wherein the cells are selected from the group consisting of mammalian embryonic stem cells, mammalian induced pluripotent stem cells, mammalian naive pluripotent stem cells, cells differentiated from mammalian embryonic stem cells, mammalian induced pluripotent stem cells and mammalian naive pluripotent stem cells, mammalian cells reprogrammed from other cell types, mammalian primary cells, human umbilical vein endothelial cells, cancer cells, T cells, mammalian tissue stem cells, mammalian cell lines, insert cells, plant cells, yeast and bacterial cells.

4. The method of claim 1 further comprising releasing the cultured cells from the hollow space of alginate hydrogel fibers comprising dissolving the alginate hydrogel fibers.

5. The method of claim 4 wherein dissolving the alginate hydrogel fibers comprises chemically dissolving the fibers using a chemical dissolvent selected from the group consisting of ethylenediaminetetraacetic acid (EDTA), ethylene glycol tetraacetic acid (EGTA), and an alginate lyase solution.

6. The method of claim 4 wherein dissolving the alginate hydrogel fibers comprises physically dissolving the fiber using a mechanical force.

7. A method of manufacturing cells at various scales, the method comprising:
extruding a cell solution and an alginate solution into a cell compatible solution, the cell compatible solution crosslinking the alginate polymers within the alginate polymer solution to form hollow alginate hydrogel fibers;
suspending the fibers including the cells in cell culture medium or cell compatible buffer; and
culturing the cells.

8. The method of claim 7 wherein the alginate solution is prepared by suspending alginate polymers in a solution at a concentration of from about 0.01% to about 20% by weight/volume alginate polymers.

9. The method of claim 7 wherein the cell compatible solution comprises one or more of calcium ions and barium ions.

10. The method of claim 7 further comprising releasing the cultured cells from the alginate hydrogel fibers comprising dissolving the alginate hydrogel fibers.

11. The method of claim 10 wherein dissolving the alginate hydrogel fibers comprises chemically dissolving the alginate hydrogel fibers using a chemical dissolvent selected from the group consisting of ethylenediaminetetraacetic acid (EDTA), ethylene glycol tetraacetic acid (EGTA), and an alginate lyase solution.

12. The method of claim 10 wherein dissolving the alginate hydrogel fiber comprises physically dissolving the alginate hydrogel fiber using a mechanical force.

13. A system for culturing cells, the system comprising:
a housing comprising a first inlet, a second inlet, a core channel, a shell channel, and an outlet, the first inlet operable for introducing a cell solution into the core channel, the second inlet operable for introducing an alginate solution into the shell channel, wherein the shell channel is in fluid contact with the core channel to allow contact between the cell solution and alginate solution, and
a cell culture vessel in fluid contact with the housing at the outlet, wherein the cell culture vessel comprises a cell compatible buffer.

14. The system of claim 13, wherein the housing further comprises a third inlet operable for introducing the alginate solution into a second shell channel.

15. The system of claim 13, wherein the cell solution comprises cells selected from the group consisting of mammalian embryonic stem cells, mammalian induced pluripotent stem cells, mammalian naive pluripotent stem cells, cells differentiated from mammalian embryonic stem cells, mammalian induced pluripotent stem cells and mammalian naive pluripotent stem cells, mammalian cells reprogrammed from other cell types, mammalian primary cells, human umbilical vein endothelial cells, cancer cells, T cells, mammalian tissue stem cells, mammalian cell lines, insert cells, plant cells, yeast and bacterial cells.

16. The system of claim 13, wherein the alginate solution comprises alginate polymer material selected from the group consisting of alginate acid polymers, sodium alginate polymers, modified alginate polymers, and combinations thereof.

17. The system of claim 13, wherein the alginate solution comprises from about 0.01% (w/v) to about 20% (w/v) alginate.

18. The system of claim 13, wherein the cell compatible buffer comprises at least one of calcium ions and barium ions.

19. The system of claim 18, wherein the cell compatible buffer comprises at least one of CaCl₂ and BaCl₂.

20. The system of claim 13, further comprising at least one pump in fluid connection with one of the first inlet and second inlet.
